SEQUENCE LISTING (7 sequences)

SEQ ID NO: 1 - DNA, 8 bp, artificial
  oligonucleotide synthesized by solid state synthesis
ttcccctt

SEQ ID NO: 2 - DNA, 10 bp, artificial
  oligonucleotide synthesized with solid state synthesis
ttcccccctt

SEQ ID NO: 3 - DNA, 8 bp, artificial
  oligonucleotide synthesized with solid state synthesis, purified then modified
ttcccctt

SEQ ID NO: 4 - DNA, 10 bp, artificial
  oligonucleotide synthesized with solid state synthesis, purified, modified
ttcccccctt

SEQ ID NO: 5 - DNA, 10 bp, artificial
  oligonucleotide synthesized with sold state synthesis
ttcccccctt

SEQ ID NO: 6 - DNA, 8 bp, artificial
  oligonucleotide synthesized with solid state synthesis
ttcccctt

SEQ ID NO: 7 - DNA, 10 bp, artificial
  oligonucleotide synthesized by solid state synthesis
ttcccccctt

(19) United States
(12) Patent Application Publication
(10) Pub. No.: US 2010/0290992 A1
(43) Pub. Date: Nov. 18, 2010

(54) NANOPARTICLE NUCLEIC ACID BINDING COMPOUND CONJUGATES FORMING I-MOTIFS

(76) Inventors: Frank Seela, Osnabrueck (DE); Simone Budow, Hasbergen (DE); Peter Leonard, Muenster (DE)

Correspondence Address: DINSMORE & SHOHL, LLP;FIFTH THIRD CENTER, ONE SOUTH MAIN STREET, SUITE 1300, DAYTON, OH, 45402, US

(21) Appl. No.: 12/367,827

(22) Filed: Feb. 9, 2009

Related U.S. Application Data
(63) Continuation of application No. PCT/EP2007/007109, filed on Aug. 10, 2007.

(30) Foreign Application Priority Data
Aug. 11, 2006 (GB) 0615961.0

(51) Int. Cl. A61K 49/00; C07D 237/06; C07D 251/08; C07D 257/08; C07D 487/04; C07D 471/04; C07D 239/00; C07H 21/04; C07H 21/02; G01N 33/566; G01N 21/00; A61K 39/385; A61K 31/7088; C40B 30/04; B05D 5/00; B05D 5/12; B05D 3/00; C12P 1/00; G01N 33/50; C12Q 1/68; A61P 37/04; G01Q 60/24; H01J 37/28

(52) U.S. Cl. 424/9.1; 544/224; 544/220; 544/179; 544/263; 544/350; 546/117; 544/255; 544/184; 536/23.1; 436/501; 436/164; 424/193.1; 514/44 R; 506/9; 427/256; 427/98.4; 427/98.5; 427/2.1; 427/2.26; 435/41; 436/94; 435/6; 850/33; 250/307

(57) ABSTRACT

The present invention concerns the field of nanoparticle bioconjugates which form an i-motif or an i-motif related structure (compositions) without or with at least one further nucleic acid binding compound. The i-motif base pairs can be charged or non-charged. Their assembly can be controlled by the pH value or temperature. At least one of these nucleic acid binding compounds has to be attached at least to a nanoparticle. The methods provide compositions used for DNA driven programmable nanoparticle assemblies, electronic circuits, diagnostic detection tools, biosensors, memory storage devices, diagnostic devices for biomolecule sequencing and detection, drug delivery, application in tumour diagnostics and treatment, nanomachines, nanofabrication, nanocatalysis, nanoarrays, and nanoscaled enzyme reactors.

### RELATED APPLICATIONS

This application is a continuation of PCT/EP2007/007109 filed Aug. 10, 2007 and claims priority to GB 0615961.0 filed Aug. 11, 2006.

### FIELD OF THE INVENTION

The present invention concerns the field of nanoparticle conjugates that form an i-motif or an i-motif related structure.

### BACKGROUND

Gold nanoparticles are one of the chemically most stable metal species allowing surface modification.

In 1676 J Kunckel concluded that in aqueous gold solutions gold must be present in such a degree of communition that the gold particles in the aqueous gold solutions are not visible to the human eye (see M.-C. Daniel, D. Astruc, Chem. Rev. 2004, 104, 293). A solution of deep red colloidal gold was prepared by the reduction of chloroaurate (AuCl₄⁻) using phosphorous by M. Faraday in 1857.

Recent advances have led to the development of functionalized nanoparticles being covalently linked to biological molecules such as nucleic acids, peptides and proteins (as reported in C. A. Mirkin, R. L. Letsinger, R. C. Mucic, J. J. Storhoff, Nature 1996, 382, 607; R. Elghanian, J. J. Storhoff, R. C. Mucic, R. L. Letsinger, C. A. Mirkin, Science 1997, 277, 1078; C. M. Niemeyer, B. Ceyhan, P. Hazarika, Angew. Chem. Int. Ed. 2003, 42, 5766; S. Chah, M. R. Hammond, R. N. Zare, Chemistry & Biology 2005, 12, 323.). One of the most successful approaches is the DNA gold nanoparticle system which has been used to construct a variety of highly ordered nano-assemblies (see C. M. Niemeyer, B. Ceyhan, P. Hazarika, Angew. Chem. Int. Ed. 2003, 42, 5766; R. C. Mucic, J. J. Storhoff, C. A. Mirkin, R. L. Letsinger, J. Am. Chem. Soc. 1998, 120, 12674; F. Seela, A. M. Jawalekar, L. Chi, D. Zhong, Chem. Biodiv. 2005, 2, 84; F. Seela, A. M. Jawalekar, L. Chi, D. Zhong, H. Fuchs, Nucleosides Nucleotides Nucleic Acids 2005, 24, 843; F. Seela, A. M. Jawalekar, L. Chi, D. Zhong ‘NanoBio NRW’, Münster, 2004).

The DNA-gold nanoparticle conjugate concept is based on the combination of the favourable properties of the gold nanoparticles and the DNA molecules to form a DNA-gold nanoparticle assembly. DNA represents a powerful molecular recognition system leading to self-assembly. The stiff structure of the DNA and the simple synthesis of DNA structures by automated DNA synthesis make it ideal for the construction of nanodevices, as has been reported in N. C. Seeman, Nature 2003, 421, 427. The DNA-gold nanoparticle assembly can be used in the bottom-up strategy of nanotechnology. The DNA-gold nanoparticle assembly is not limited to single-stranded or duplex DNA but can also incorporate higher ordered DNA structures such as triplexes, quadruplexes and pentaplexes that are readily formed depending on particular sequence motifs (D. E. Gilbert, J. Feigon, Curr. Opin. Struc. Biol. 1999, 9, 305).

US Patent Application No 2006/0068378 (Mirkin et al.) has disclosed the use of a gold nanoparticle-oligonucleotide conjugate as a means of detecting nucleic acids. This involves the selection of an oligonucleotide sequence complementary to the sequence of the nucleic acid. The nucleic acid “bridges” the two nanoparticle-oligonucleotide conjugates, thus aggregating the nanoparticle-oligonucleotide conjugate. The aggregation can be detected by scattered light.

Repetitive DNA sequences which are interspersed throughout the human genome are capable of folding into a variety of complex structures. Cytosine-rich regions such, as the centromer and telomer domains as well as the insulin mini-satellite are assumed to form a unique tetrameric structure which is designated as i-motif (see J.-L. Leroy, M. Guéron, J.-L. Mergny, C. Hélène, Nucleic Acids Res. 1994, 22, 1600; P. Catasti, X. Chen, L. L. Deaven, R. K. Moyzis, E. M. Bradbury, G. Gupta, J. Mol. Biol. 1997, 272, 369; M. Guéron, J.-L. Leroy, Curr. Opin. Struc. Biol. 2000, 10, 326; A. T. Phan, M. Guéron, J.-L. Leroy, J. Mol. Biol. 2000, 299, 123; A. T. Phan, J.-L. Mcrgny, Nucleic Acids Res. 2002, 30, 4618). The i-motif consists of two sets of paired duplexes containing stretches of cytosine residues to form a quadruplex as is shown in FIG. 17. The two sets of paired duplexes are stabilized by hemiprotonated non-canonical cytosine-cytosine base pairs in which a protonated dC⁺ is situated opposite to an unprotonated dC residue with parallel chain orientation of the phosphodiester backbone (see FIG. 17). Two of these duplexes are associated in an antiparallel way by base-pair intercalation (see FIG. 17). The cytosine residues have a right-handed twist of 17-18°. The i-motif displays two wide and two narrow grooves with close sugar contacts. Crystal structures of the intercalated i-motif have been reported in C. H. Kang, I. Berger, C. Lockshin, R. Ratliff, R. Moyzis, A. Rich, Proc. Natl. Acad. Sci. USA 1994, 91, 11636 and I. Berger, M. Egli, A. Rich, Proc. Natl. Acad. Sci. USA 1996, 93, 12116. Consistent with the hemiprotonation of the cytosine residues the i-motif assembly is formed under weak acidic conditions (pH=5.5) (J.-L. Mergny, L. Lacroix, X. Han, J.-L. Leroy, C. Hélène, J. Am. Chem. Soc. 1995, 117, 8887; L. Chen, L. Cai, X. Zhang, A. Rich, Biochemistry, 1994, 33, 13540; K. Gehring, J.-L. Leroy, M. Guéron, Nature 1993, 363, 561.

Recently, the synthesis and properties of multiple-stranded DNA-gold conjugates using the ion-specific aggregation of the dG quartet hairpin 5′-d(G₄T₄G₄) were reported in F. Seela, A. M. Jawalekar, L. Chi, D. Zhong, Chem. Biodiv. 2005, 2, 84 and F. Seela, A. M. Jawalekar, L. Chi, D. Zhong, H. Fuchs, Nucleosides Nucleotides Nucleic Acids 2005, 24, 843. These observations have been used later by others for the same purpose (Z. Li, C. A. Mirkin, J. Am. Chem. Soc. 2005, 127, 11568).

The pH-dependent assembly of DNA modified nanoparticles on the basis of i-motifs or i-motif related structures offers the opportunity to design DNA driven programmable nanoparticle assemblies, electronic circuits, diagnostic detection tools, biosensors, memory storage devices, diagnostic devices for biomolecule sequencing and detection, drug delivery, application in tumour diagnostics and treatment, nanomachines, nanofabrication, nanocatalysis, nanoarrays and nanoscaled enzyme reactors.

### SUMMARY OF THE INVENTION

In summary, the present invention discloses compositions which consist of an i-motif structure or an i-motif related structure and comprise at least one nanoparticle. The i-motif structure or i-motif related structure is formed by at least one bioconjugate and (i) without or (ii) with at least one further nucleic acid binding compound. The composition is used for various methods in the fields of diagnostic, detection and surface chemistry.

The base pairs forming the i-motif structure or the i-motif related structure can be charged or non-charged. The assembly of the i-motif structure or the i-motif related structure to form a composition can be controlled by the pH value or temperature.

The present invention also discloses methods for DNA driven programmable nanoparticle assemblies, electronic circuits, diagnostic detection tools, biosensors, memory storage devices, diagnostic devices for biomolecule sequencing and detection, drug delivery, application in tumour diagnostics and treatment, nanomachines, nanofabrication, nanocatalysis, nanoarrays and nanoscaled enzyme reactors.

### BRIEF DESCRIPTION OF THE FIGURES

FIG. 1 shows CD-spectra of the i-motif assembly 5′-d(T₂C₄T₂) (1) measured in 0.3 M NaCl, 10 mM phosphate buffer at various temperatures under acidic conditions [pH 5.5; (a)] and under alkaline conditions [pH 8; (b)].

FIG. 2 shows CD-spectra of the i-motif construct 5′-trityl-S—(CH₂)₆—O(PO₂H)O-d(TTC CCC CCT T) (4) measured in 10 mM sodium phosphate buffer containing 0.3 M NaCl at pH 5.5 (a) and the single-stranded spiecies at pH 8.0 (b) measured at various temperatures.

FIG. 3 shows UV/VIS spectra of the alkaline solution (pH=9.5) of 15 nm diameter gold nanoparticles (a). Gold nanoparticles functionalized with 5′-(sulfanylhexanyl)-d(TTC CCC CCT T) (4) measured in 10 mM phosphate buffer, pH=8.0 containing 0.3 M NaCl (b) and Au nanoparticles functionalized with 4 measured in 10 mM phosphate buffer, pH=5.0 containing 0.3 M NaCl after i-motif formation.

FIG. 4 shows a schematic representation of the assembly of bioconjugate 5; T=dT and C=dC.

FIG. 5 shows the colour change of the solution of the DNA-gold conjugate 5 in 10 mM phosphate buffer containing 0.1 M NaCl (left: pH=5.5; right: pH=6.5).

FIG. 6 shows multiple working-cycles of the nanomachine in 10 mM phosphate buffer with 0.1 M NaCl. The cyclic absorption changes were induced by repetitive addition of 1M HCl or 1M NaOH. The absorbance was corrected by a factor resulting from dilution with acid and base ( . . . →—).

FIG. 7 shows the pKₐ-values of 2’-deoxycytidine (7) and 5-propynyl-2′-deoxycytidine (8).

FIG. 8 shows the hemiprotonated base pairs of 5-propynyl-2′-deoxycytidine (8).

FIG. 9 shows (a) CD-spectra of 9 measured in 0.3 M NaCl, 10 mM phosphate buffer at various temperatures under acidic conditions (pH 5) after an incubation time of 20 days and (b) after formation of the i-motif in 0.3 M NaCl, 10 mM phosphate buffer, pH 3.3 at various temperatures (incubation time 18 h).

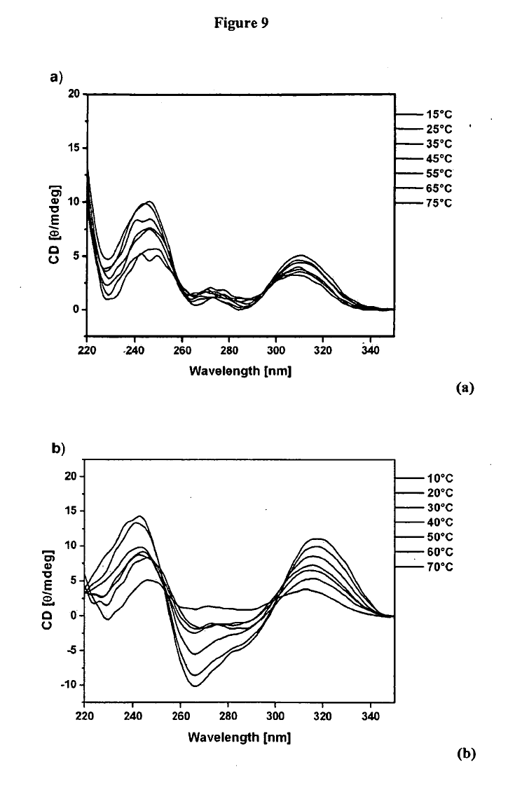

FIG. 10 shows UV/VIS spectra of (a) the alkaline solution (pH=9) of 15 nm diameter gold nanoparticles. Bioconjugate 12 measured in 10 mM phosphate buffer containing 0.1 M, NaCl at (b) pH=7.0 as disperse system and (c) at pH=2.5 after formation of the composition.

FIG. 11 shows the schematic representation of the assembly of bioconjugates.

FIG. 12 shows pH-dependent UV/VIS-spectra of the Au-DNA nanoparticle conjugates 6 (A) and 11 (B) measured in 0.1 M NaCl, 10 mM phosphate buffer at various pH-values.

FIG. 13 shows UV/VIS absorption change induced by the addition of 20 μl 1M HCl to bioconjugate 5 (A) and 11 (B) measured in 0.1 M NaCl, 10 mM phosphate buffer.

FIG. 14 shows the synthesis route of compound 20.

FIG. 15 shows the synthesis route of compound 24.

FIG. 16 shows the synthesis routes of compounds 29a and 29b.

FIG. 17 shows the i-motif assembly stabilized by hemiprotonated cytosine base-pairs.

### DETAILED DESCRIPTION OF THE INVENTION

The subject of the present invention discloses a composition consisting of at least one bioconjugate which forms an i-motif or an i-motif related structure (i) without or (ii) with at least one further nucleic acid binding compound. Further, the present invention discloses methods for uses of the bioconjugates which are based on such compositions.

Terms and Definitions

Conventional techniques of molecular biology and nucleic acid chemistry, which are within the skill of the art, are fully explained in the literature. See, for example, Sambrook et al., 1989, Molecular Cloning—A Laboratory Manual, Cold Spring Harbor Laboratory, Cold Spring Harbor, N.Y.; Oligonucleotide Synthesis (M. J. Gait, ed., 1984); Nucleic Acid Hybridization (B. D. Hames and S. J. Higgins. Eds., 1984); and a series, Methods in Enzymology (Academic Press, Inc.), all of which are incorporated herein by reference. All patents, patent applications, and publications mentioned herein, both supra and infra, are incorporated herein by reference.

The terms “nucleic acid” and “oligonucleotide” or “polynucleotide” refer to polydeoxyribonucleotides (containing 2-deoxy-D-ribose), to polyribonucleotides (containing D-ribose), and to any other type of polynucleotide which is a C or N glycoside of a purine or pyrimidine base, modified purine or pyrimidine base or any other heterocycle. The sugar moiety is not limited to D- or L-ribose; other sugars known to men skilled in the art are also included. Also, the phosphodiester linkage can be modified. Typical examples are the phosphorothioates. There is no intended distinction of the chain length between the terms “nucleic acid” and “oligonucleotide”, and these terms will be used interchangeably. These terms refer only to the primary structure of the molecule. Thus, these terms include double- and single-stranded nucleic acids as well as more complex structures such as triplexes, quadruplcxes and higher assemblies are included.

The term “backbone” or “nucleic acid backbone” for a nucleic acid binding compound according to the invention refers to the structure of the chemical moiety linking nucleobases in a nucleic acid binding compound. The nucleobases are attached to the backbone and take part in base pairing to other nucleic acid binding compounds via hydrogen bonding and/or base stacking. This may include structures formed from any and all means of chemically linking nucleotides, e.g., the natural occurring phosphodiester ribose backbone or unnatural linkages, e.g., phosphorothioates, methyl phosphonates, phosphoramidates and phosphotriesters. Peptide nucleic acids have unnatural linkages. Therefore, a “modified backbone” as used herein includes modifications to the chemical linkage between nucleotides as described above, as well as other modifications that may be used to enhance stability and affinity, such as modifications to the sugar structure. For example, an α-anomer of deoxyribose may be used, where the base is inverted with respect to the natural β-anomer. In an embodiment, the 2′-OH of the sugar group may be altered to 2′-O-alkyl, which provides resistance to degradation without comprising affinity.

The term “nucleic acid binding compound” refers to substances which associate with other nucleic acid binding compounds of any sequence which are able to function as binding partner. The binding preferably occurs via hydrogen bonding and/or stacking between base pairs. Non-natural bases attached to the backbone of the nucleic acid binding compound are also involved in these interactions. The expert in the field recognizes that the most well-known “nucleic acid binding compounds” are nucleic acids.

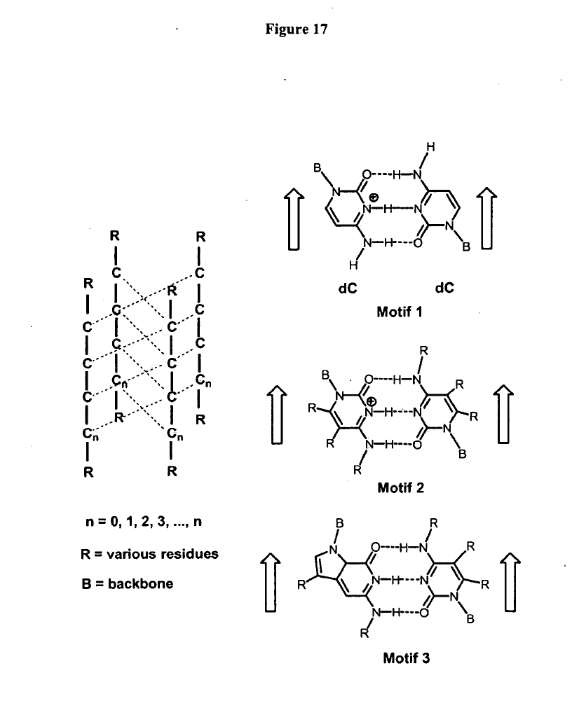

The term “i-motif' refers to a structure that consists of two sets of parallel paired duplexes containing stretches of cytosine residues to form a quadruplex as it is shown in FIG. 17. The two sets of paired duplexes are stabilized by hemiprotonated non-canonical cytosine-cytosine base pairs in which a protonated dC⁺ is situated opposite to an unprotonated dC residue with parallel chain orientation of the phosphodiester backbone (see FIG. 17). Two of these duplexes are associated in an antiparallel way by base-pair intercalation (see FIG. 17). The cytosine residues have a right-handed twist of 17-18°. The i-motif displays two wide and two narrow grooves with close sugar contacts. Crystal structures of the intercalated i-motif have been reported in C. H. Kang, I. Berger, C. Lockshin, R. Ratliff, R. Moyzis, A. Rich, Proc. Natl. Acad. Sci. USA 1994, 91, 11636 and I. Berger, M. Egli, A. Rich, Proc. Natl. Acad. Sci. USA 1996, 93, 12116. Consistent with the hemiprotonation of the cytosine residues the i-motif assembly is formed under weak acidic conditions (pH=5.5) (J.-L. Mergny, L. Lacroix, X. Han, J.-L. Leroy, C. Helene, J. Am. Chem. Soc. 1995, 117, 8887; L. Chen, L. Cai, X. Zhang, A. Rich, Biochemistry, 1994, 33, 13540; K. Gehring, J.-L. Leroy, M. Guéron, Nature 1993, 363, 561. The term “i-motif” includes structures which are related to the i-motif. Also, i-motif structures or i-motif related structures containing modifications in the heterocycle or the backbone are included. Thus, i-motif related structures can also be formed by nucleic acid binding compounds and any further modified nucleic acid binding compound exhibiting one kind or more than one kind of cytosine analogue showing the donor/acceptor pattern of cytosine (examples see formulae 1-5). The i-motif structure or i-motif related structure is stabilized by hemiprotonated base-pairs in analogy to the hemiprotonated cytosine-cytosine base-pair or by non-charged base pairs.

The term “nanoparticle” refers to a microscopic particle whose size is measured in nanometers (nm). It is defined as a particle with at least one dimension which is less than 200 nm. Nanoparticles made of semiconducting material may also be labeled quantum dots if they are small enough (typically less than 10 nm) that quantization of electronic energy levels occurs. Nanoparticles often have unexpected physical or chemical properties. They are small enough to scatter visible light rather than absorb it. Depending on the particle size gold nanoparticles appear deep red to black in solution.

The term “bioconjugate” refers to a construct in which the nucleic acid binding compound is linked to a nanoparticle. The bioconjugate exhibits the ability to form an i-motif structure or an i-motif related structure.

The term “composition” refers to an assembly of at least one bioconjugate (i) without or (ii) with at least one further nucleic acid binding compound. This assembly contains at least one i-motif structure or i-motif related structure.

The term “polarity” refers to the direction of a chain, e.g., a nucleic acid, a peptide or another structure. In nucleic acids the change of the polarity means a change from 5′→3′ to 3′→5′.

The terms “parallel” and “antiparallel” chain orientation describe the orientation of the polarity of two or more chains, e.g., oligonucleotide chains, to each other.

The term “nanomachine” refers to mechanical devices having nanometer dimensions. Nanomachines are found in nature, but are also built synthetically for applications in medicine, computer science or nanobiotechnology. Nanomachines are capable of rotation, stretching, vibration and movement motions, etc.

The term “array” describes a substrate of a defined material and structure. “Nanoscopic array” or “nanoarray” refers to an array of nanometer dimensions.

The term “nanoscopic device” refers to an object of nanometer dimensions and any shape, e.g., nanoarray, nanoassembly, nanomachine, sensor, wire etc.

The term “nanoscopic switch” refers to nanodevices which change their properties between two or more states by internal and/or externals signals.

The term “nanoassembly” refers to nanostructured materials aggregated from previously prepared nanobuilding blocks, e.g., from nanoparticles which form an i-motif structure. The nanoassemblies are formed by self-assembly.

The term “network” refers to highly organized systems, e.g., films, stable colloids, gels, fibres which may have the capability to form pores for the inclusion of other molecules.

The term “stabilizer” refers to compounds which increase duplex, triplex or tetraplex stability by using modified heterocycles or modified backbones. Stability can also be increased with drugs or dyes, e.g., actinomycin or ethidiumbromide.

“Reporter groups” are generally groups that make the nucleic acid binding compound as well as any nucleic acid bound thereto distinguishable from the remainder of a liquid, i.e., the sample (nucleic acid binding compounds having attached a reporter group can also be termed labelled nucleic acid binding compound). The term “reporter group” and the specific embodiments preferably include a linker which is used to connect the moiety to the reporter group. The linker will provide flexibility such that the nucleic acid binding compound can bind the nucleic acid sequence to be identified. Linkers, especially those that are not hydrophobic, for example, based on consecutive ethylenoxy units, for example, as disclosed in DE 3943522, are known to person skilled in the art.

The term “protecting group” refers to a chemical group that is attached to a functional moiety (for example, to the oxygen in a hydroxyl group or the nitrogen in an amino group, replacing the hydrogen) to protect the functional group from reacting in an undesired way. A protecting group is further defined by the fact that it can be removed without destroying the biological activity of the molecule formed. Suitable protecting groups are known to a man skilled in the art. The protecting groups include, but are not limited to hydroxyl groups at the 5′-end of a nucleotide or oligonucleotide are selected from the trityl groups, for example, dimethoxytrityl.

Preferred protecting groups at exocyclic amino groups of the heterocycles in formulae 1-5 are the acyl groups, most preferred the benzoyl group (Bz), phenoxyacetyl or acetyl or formyl, and the N,N-dialkylformamidine group, preferentially the dimethyl-, diisobutyl-, and the di-n-butylformamidine group.

Preferred O-protecting groups are the aroyl groups, the diphenylcarbamoyl group, the acyl groups, the silyl groups and photoactivable groups as ortho nitro-benzyl protecting groups like 2-(4-nitrophenyl)ethoxycarbonyl (NPEOC). Among these most preferred is the benzoyl group.

Preferred silyl groups are the trialkylsilyl groups, like, trimethylsilyl, triethylsilyl and tertiary butyl-dimethyl-silyl. Another preferred silyl group is the trimethylsilyl-oxy-methyl group (TOM) (Swiss Patent Application 01931/97).

During chemical synthesis, any groups —OH, —SH and —NH₂ (including those groups in reporter groups) should be protected by suitable protecting groups.

Halogen means a fluoro, chloro, bromo or iodo group.

Alkyl groups are preferably chosen from alkyl groups containing from 1 to 50 carbon atoms, either arranged in linear, branched or cyclic form. The actual length of the alkyl group will depend on the steric situation at the specific position where the alkyl group is located. If there are steric constraints, the alkyl group will generally be smaller, the methyl and ethyl group being most preferred. All alkyl, alkenyl and alkynyl groups can be either unsubstituted or substituted. Substitution by hetero atoms will help to increase solubility in aqueous solutions.

Alkenyl groups are preferably selected from alkenyl groups containing from 2 to 50 carbon atoms. For the selections similar considerations apply as for alkyl groups. The alkenyl groups can be linear, branched and cyclic. The alkenyl groups can contain more than one double-bond.

Alkynyl groups have preferably from 2 to 50 carbon atoms. Again, those carbon atoms can be arranged in linear, branched and cyclic manner. Further, there can be more than one triple bond in the alkynyl group.

Alkoxy groups preferably contain from 1 to 50 carbon atoms and are attached to the rest of the moiety via the oxygen atom. For the alkyl group contained in the alkoxy groups, the same considerations apply as for alkyl groups.

By “aryl” and “heteroaryl” (or “heteroaromatic”, “heterocycle”) is meant a carbocyclic or heterocyclic group comprising at least one ring having physical and chemical properties resembling compounds such as an aromatic group of 5 to 6 ring atoms and comprising 4 to 20 carbon atoms, usually 4 to 9 or 4 to 12 carbon atoms, in which one to three ring atoms is N, S or O, provided that no adjacent ring atoms are O—O, S—S, O—S or S—O. Aryl and heteroaryl groups include phenyl, 2-, 4- and 5-pyrimidinyl, 2-, 4- and 5-thiazoyl, 2-s-triazinyl, 2-, 4-imidazolyl, 2-, 4- and 5-oxazolyl, 2-, 3- and 4-pyridyl, 2- and 3- thienyl, 2- and 3-furanyl, 2- and 3-pyrrolyl optionally substituted preferably on a ring C by oxygen, alkyl of 1-4 carbon atoms or haloalkyl of 1-4 carbon atoms and 1-4 halogen atoms. Exemplary substituents on the aryl or heteroaryl group include benzyl and the like. “Heteroaryl” also means systems having two or more rings, including bicycle moieties such as benzimidazole, benzotriazole, benzoxazole; and indole. Aryl groups are the phenyl or naphtyl moiety, either unsubstituted or substituted by one more of amino, -aminoalkyl, —O—(C₁-C₁₀)-alkyl, —S—(C₁-C₁₀)-alkyl, - (C₁-C₁₀)-alkyl, sulfonyl, sulfenyl, sulfinyl, nitro and nitroso. Most preferred aryl group is the phenyl group. Preferred arylalkyl group is the benzyl group. The preferred alkylamino group is the ethylamino group. The preferred —COO (C₁-C₄) alkyl group contains one or two carbon atoms in the alkyl moiety (methyl or ethyl esters). Other aryl groups are heteroaryl groups as, e.g., pyrimidine, purine, pyrrol, or pyrazole.

Aryloxy groups preferably contain from 6 to 50 carbon atoms. Those carbon atoms may be contained in one or more aromatic rings and further in side chains (for example, alkyl chains) attached to the aromatic moiety. Preferred aryloxy groups are the phenoxy and the benzoxy group.

Any atom in the definitions within the formulae presented herein is not limited to a specific isotope. Thus, a phosphorous atom (F) can either mean the regular ³¹P or the radioactive ³²P or a mixture thereof. The same applies for any atom, e.g., hydrogen (H/D/T), carbon (C), iodine (Cl, Br, I) and nitrogen (N).

Modifications of the Heterocycles within the i-motif

The composition comprises at least two cytidine residues. In addition the compositions are capable of incorporating at least one modified cytosine residues. Non-limiting examples (Formulae 1-5) of the modified cytosine residues include:

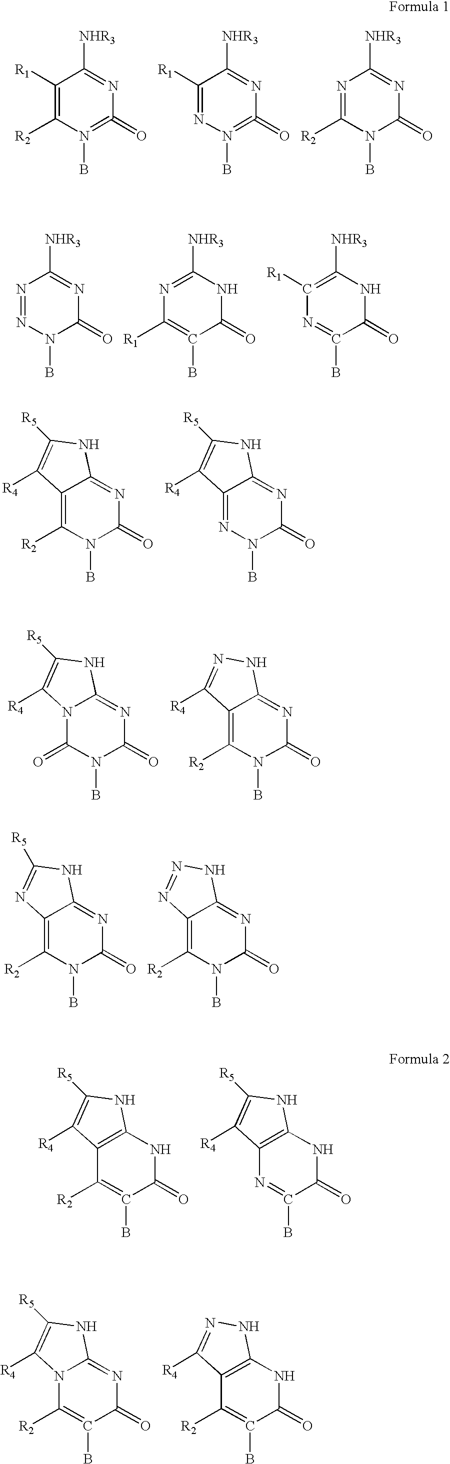

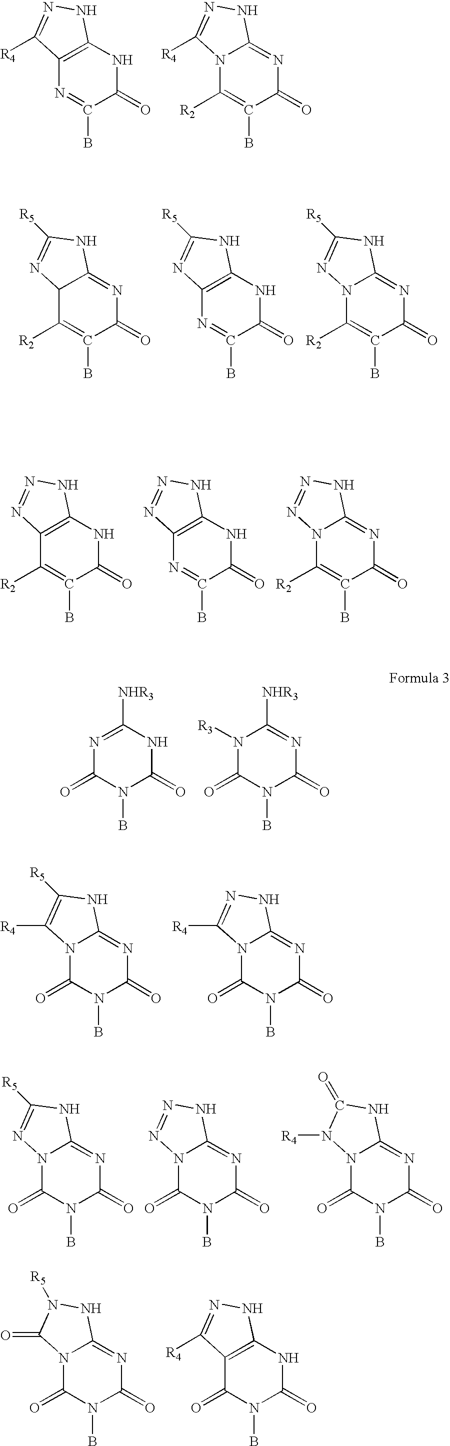

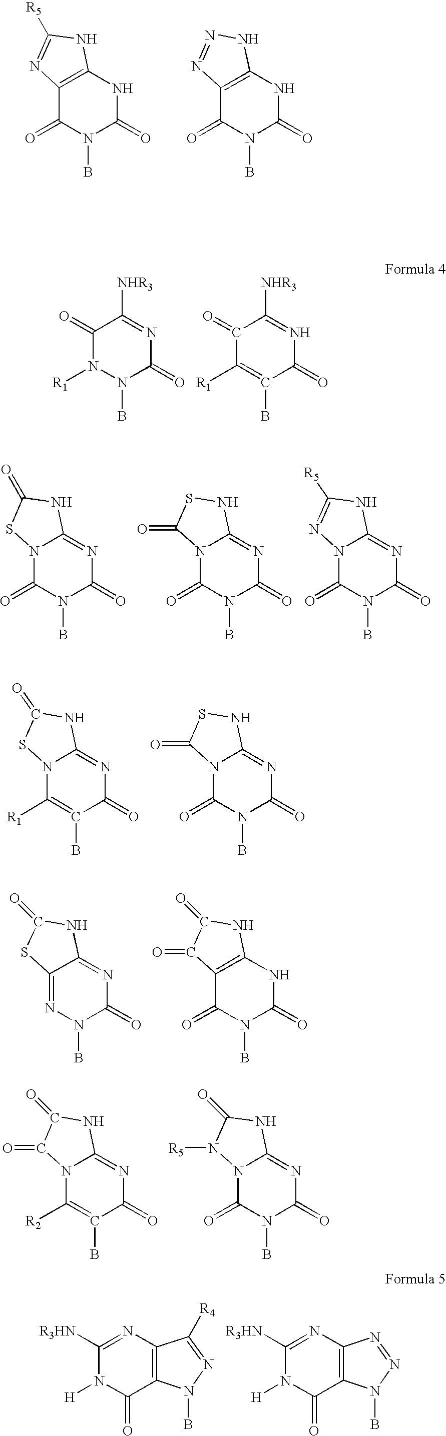

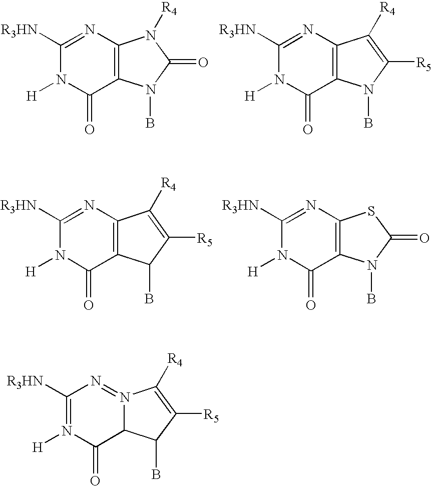

wherein

R₁, R₂, R₄, and R₅ are independent from each other and they are independent from R₃;

R₁, R₂, R₄, and R₅ are selected from the group consisting of
(1) —H,
(2) —F, —Cl, —Br, or —I,
(3) Nitro,
(4) Amino,
(5) Cyano,
(6) —COO⁻,
(7) (C₁-C₅₀)-alkyl substituted according to (12),
(8) (C₂-C₅₀)-alkenyl substituted according to (12),
(9) (C₂-C₅₀)-alkynyl substituted according to (12),
(10) (C₆-C₅₀)-aryl substituted according to (12),
(11) —W—(C₁-C₅₀)-alkyl, —W—(C₁-C₅₀)-alkenyl, —W—(C₁-C₅₀)-alkynyl, —W—(C₆-C₅₀)-aryl or W—H, wherein W=—S—, —O—, —NH—, —S—S—, —CO—, —COO—, —CO—NH—, —NH—, —NH—CO—NH—, NH—CS—NH—, —(CH₂)ₙ-[O—(CH₂)ᵣ]ₛ—, where r and s are, independently of each other, an integer between 1 to 18 and n is 0 or 1 independently from r and s,
(12) substituents (7) to (11) wherein any alkyl, alkenyl, alkynyl or aryl can be substituted by one or more moieties selected from the group consisting of -halogen, —SH, —NO₂, —CN, —S—(C₁-C₆)-alkyl, —(C₁-C₆)-alkoxy, —OH, NR⁶R⁷, —N⁺R⁶R⁷R⁸, —OR¹², —COR⁹, —NH—CO—NR⁶R⁷, —NH—CS—NR⁶R⁷, and —(CH₂)ₙ-[O—(CH₂)ᵣ]ₛ—NR⁶R⁷ where r and s are, independently of each other, an integer between 1 to 18 and n is 0 or 1 independently from r and s, wherein R⁹ is selected from the group consisting of —OH, —(C₁-C₆)-alkoxy, —(C₆-C₂₂)-aryloxy, —NHR⁸, —OR⁸, —SR⁸, wherein R⁶, R⁷, and R⁸ are selected independently from the group consisting of —H, —(C₁-C₁₀)-alkyl, —(C₁-C₁₀)-alkenyl, —(C₁-C₁₀)-alkynyl, —(C₆-C₂₂)-aryl and a reporter group or a group which facilitates intracellular uptake said alkyl, alkenyl, alkynyl or aryl in substituents (7) to (12) being unsubstituted or substituted by one or more moieties selected from the group consisting of -halogen, —SH, —S—(C₁-C₆)-alkyl, —(C₁-C₆)-alkoxy, —OH, NR⁶R⁷, —COR⁹, —NH—CONR⁶R⁷, —NH—CSNR⁶R⁷, and —(CH₂)ₙ-[O—(CH₂)ᵣ]ₛ—NR⁶R⁷ where r and s are, independently of each other, an integer between 1 to 18 and n is 0 or 1 independently from r and s, with the proviso that R⁶, R⁷ or R⁸ is not a reporter group if the radicals (7) to (9) are substituted by —NR⁶R⁷, —NHR⁸, —OR⁸, or —SR⁸;

R₃ is independent from R₁, R₂, R₄, or R₅ and is selected from the group of
(1) —H,
(2) (C₁-C₅₀)-alkyl,
(3) (C₂-C₅₀)-alkenyl,
(4) (C₂-C₅₀)-alkynyl,
(5) (C₆-C₅₀)-aryl,
(6) (C₆-C₅₀)-aryloxy,
(7) —Z—(C₁-C₅₀)-alkyl, —Z—(C₁-C₅₀)-alkenyl, —Z—(C₁-C₅₀)-alkynyl, —Z—(C₆-C₅₀)-aryl or Z—H, wherein Z=—CO—, —CO—NH—, —CS—NH—, —(CH₂)ₙ-[O—(CH₂)ᵣ]ₛ—, where r and s are, independently of each other, an integer between 1 to 18 and n is 1 or 2 independently from r and s,
(8) substituents (2) to (7) wherein any alkyl, alkenyl, alkynyl or aryl can be substituted by one or more moieties selected from the group consisting of -halogen, NO₂, —OR⁸, —CN, —SH, —S—(C₁-C₆)-alkyl, —(C₁-C₆)-alkoxy, —OH, NR⁶R⁷, —N⁺R⁶R⁷R⁸, —COR⁹, —NH—CONR⁶R⁷, —NH—CSNR⁶R⁷, and —(CH₂)ₙ—O—(CH₂)ᵣ]ₛ—NR⁶R⁷ where r and s are, independently of each other, an integer between 1 to 18 and n is 0 or 1 independently from r and s, wherein R⁹ is selected from the group consisting of —OH, —(C₁-C₆)-alkoxy, —(C₆-C₂₂)-aryloxy, —NHR⁸, —OR⁸, —SR⁸, wherein R⁶, R⁷, and R⁸ are selected independently from the group consisting of —H, —(C₁-C₁₀)-alkyl, —(C₁-C₁₀)-alkenyl, —(C₁-C₁₀)-alkynyl, —(C₆-C₂₂)-aryl and a reporter group, said alkyl, alkenyl, alkynyl or aryl in substituents (2) to (8) being unsubstituted or substituted by one or more moieties selected from the group consisting of -halogen, —SH, —S—(C₁-C₆)-alkyl, —(C₁-C₆)-alkoxy, —OH, NR⁶R⁷, —COR⁹, —NH—CONR⁶R⁷, —NH—CSNR⁶R⁷, and —(CH₂)ₙ-[O—(C₂)ᵣ]ₛ—NR⁶R⁷ where r and s are, independently of each other, an integer between 1 to 18 and n is 0 or 1 independently from r and s; and

B is the position of attachment of the group to the backbone of the nucleic acid binding compound and any salts thereof.

The heterocyclic groups of Formulae 1-5 are mainly characterized by the following properties:
The heterocycle is linked to a backbone, preferred to a sugar moiety, via a nitrogen or carbon.
The heterocycle contains an aromatic π-electron system which is capable of forming stacking interactions with other nucleic acid constituents.
The heterocyclic group displays the donor/acceptor pattern as it is characteristic for the natural occurring cytosine.

The present invention also contemplates tautomeric forms and salts of heterocyclic groups of Formulae 1-5.

Reporter Groups within the i-motif

The bioconjugate and any further nucleic acid binding compound which assembles to the composition can be modified at the i-motif with a reporter group which is used for a detection protocol.

While as many reporter groups can be attached as useful to label the bioconjugate and/or the nucleic acid binding compound sufficiently, it is preferred to attach only a limited number of reporter groups to a single subunit. This is to ensure that recognition and affinities of the bioconjugate and/or the nucleic acid binding compound and its solubility are not affected in such a manner that the bioconjugate and/or the nucleic acid binding compound are not able to form an i-motif structure or any i-motif related structure.

In one embodiment of the invention, there will be only 1 to 4, most preferably 1 or 2 or most preferred a single reporter group in each bioconjugate and/or nucleic acid binding compound. There are formats for the determination of nucleic acids which require more than one reporter group attached to a probe. An example for such formats is disclosed in the international patent application no WO92/02638. In the example discussed in this patent application, one of the reporter groups is a fluorescence quencher. Fluorescence quenching occurs when the fluorescent group and the fluorescence quencher are in close proximity to each other. Fluorescence occurs only when the fluorescence quencher and a fluorescent group (as the reporter group) are separated.

Reporter groups are generally groups that make the bioconjugate and/or the nucleic acid binding compound distinguishable from the remainder of a liquid (nucleic acid binding compounds having attached a reporter group can also be termed labelled nucleic acid binding compound). This distinction can be either effected by selecting the reporter group from the group of directly or indirectly detectable reporter groups or from the groups of immobilized or immobilizable groups.

Directly detectable reporter groups are, for example, fluorescent groups, such as but not limited to fluorescein and its derivatives, like hexachlorofluorescein and hexafluorofluorescein, rhodamines, psoralens squaraines, porphyrins, fluorescent particles, bioluminescent compounds, like acridinium esters and luminol, or the cyanine dyes, like Cy-5. Examples of such compounds are disclosed in the European Patent Application EP 0 680 969.

Further, spin labels like TEMPO, electrochemically detectably groups, ferrocene, viologene, heavy metal chelates and electrochemiluminescent labels, like ruthenium bispyridyl complexes, and naphthoquinones, quencher dyes, like dabcyl, and nuclease active complexes, for example, of Fc and Cu, are useful detectable groups. Other examples of such compounds are europium complexes.

Indirectly detectable reporter groups are reporter groups that can be recognized by another moiety which is directly or indirectly labelled. Examples of such indirectly detectable reporter groups include but are not limited to haptens, like digoxigenin which is detectable by means of ELISA or biotin. Digoxigenin, for example, can be recognized by antibodies against digoxigenin. Those antibodies may either be labelled directly or can be recognized by labelled antibodies directed against the (digoxigenin) antibodies. Formats based on the recognition of digoxigenin are disclosed in EP-B-0 324 474. Biotin can be recognized by avidin and similar compounds, like streptavidin and other biotin binding compounds. Again, those compounds can be labelled directly or indirectly. Further interesting labels are those directly detectable by atomic force microscopy (AFM) or scanning tunnelling microscopy (STM).

A reporter group can further be a nucleotide sequence which does not interfere with other nucleotide sequences in the sample. The sample can therefore be specifically recognized by oligonucleotides of a complementary sequence. This nucleotide sequence can therefore be labelled directly or indirectly or can be immobilizable or immobilized.

A reporter group can further be a solid phase. Nanoparticles are included in the definition of the solid phase. Attachment of the bioconjugate and/or the nucleic acid binding compound with a solid phase can be either directly or indirectly as discussed above for the detectable group. Examples of such solid phases include but are not limited to latex beads or preferred nanoparticles such as gold nanoparticles. Solid phases that are useful for the immobilization of the probe according to the invention are selected from the group of polystyrene, polyethylene, polypropylene, glass, SiO₂ and TiO₂. The formats of such solid phases can be selected according to the needs of the instrumentation and format of the assay.

In another embodiment of the invention, a further reporter group attached to the bioconjugate and/or the nucleic acid binding compound may be any positively or negatively charged group. Examples of such positively or negatively charged groups include a carboxylate group or an ammonium N⁺R⁶R⁷R⁸ groups with substituents as specified under formulae 1-5 as described above. These may be attached, e.g., via a propargylen linker to the heterocycle and enhance the sensitivity of MALDI-TOF mass spectroscopy (MALDI-TOF: matrix-assisted laser desorption/ionization time-of-flight) in the positive or negative mode. The substituents of the ammonium group are preferably introduced into the bioconjugate and/or the nucleic acid binding compound via post-labelling, i.e., bioconjugates and/or nucleic acid binding compounds can be post-labelled with reporter groups when a suitable reactive group is introduced during their synthesis. One example would be the protection of the amino group of a precursor during synthesis with a phthaloyl group.

A reporter group can further be an intercalator such as ethidiumbromide, acridinium esters or actinomycin. Typical intercalating and cross-linking residues which bind to bioconjugates and/or nucleic acid binding compounds or intercalate with them and/or cleave or cross-link them, are for example, acridine, psoralene, phenanthridine, naphthoquinone, daunomycin or chloroethylaminoaryl conjugates.

A reporter group can further be a group which favours intracellular uptake. Examples of groups which favour intracellular uptake are different lipophilic residues, such as —O—(CH₂)ₓ—CH₃, in which x is an integer from 6 to 18, —O—(CH₂)ₙ—CH═CH—(CH₂)ₘ—CH₃, in which n and m are, independently of each other, an integer from 6 to 12, —O—(CH₂CH₂O)₄—(CH₂)₉—CH₃, —O—(CH₂CH₂O)₈—(CH₂)₁₃—CH₃ and —O—(CH₂CH₂O)₇—(CH₂)₁₅—CH₃, and also steroid residues, like cholesteryl, or vitamin residues such as vitamin E, vitamin A or vitamin D, and other conjugates which exploit natural carrier systems, such as bile acid, folic acid, 2-(N-alkyl, N-alkoxy)-aminoanthraquinone and conjugates of mannose and peptides of the corresponding receptors which lead to receptor-mediated endocytosis of the oligonucleotides, such as EGF (epidermal growth factor), bradykinin, and PDGF (platelet derived growth factor).

In a general manner, the described reporter groups can be introduced either at the level of the bioconjugate and/or the nucleic acid binding compound (for example, by way of SH groups) or at the level of the monomers (phosphonates, phosphoamidites or triphosphates). In the case of the monomers, in particular in the case of the triphosphates, it is advantageous to leave the side chains, into which a reporter group or an intercalator group is to be introduced, in the protected state and only to eliminate the side-chain protective groups, and to react with an optionally activated derivative of the corresponding reporter group or intercalator group, after the phosphorylation.

Typical labelling groups include, but are not limited to:

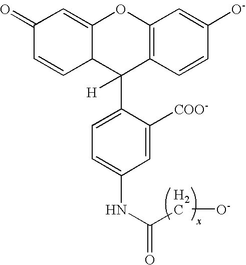

Fluorescein derivatives, where x=2-18, preferably 4

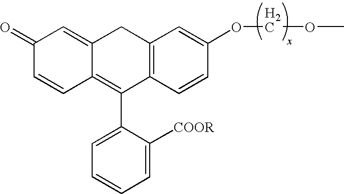

Fluorescein derivative, where R═H or C₁-C₄-alkyl

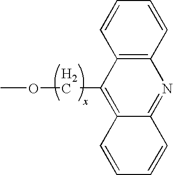

Acridine derivatives where x=2-12, preferably 4

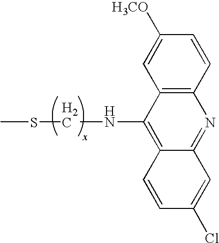

Acridine derivatives where x=2-12, preferably 4

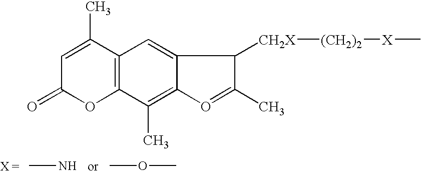

Trimethylpsoralene conjugate (=“Psoralenc” for X═O)

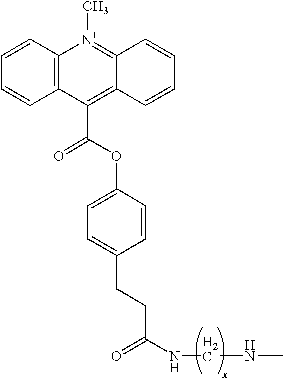

Acridinium Ester

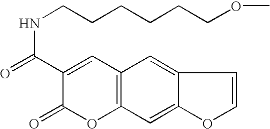

Psoralene Conjugate

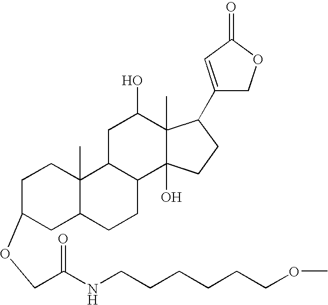

Digoxygenin conjugates

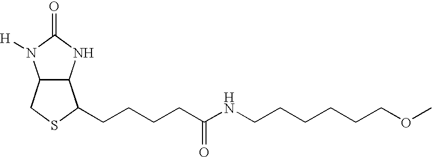

Biotin conjugate (=“Biotin” for R=Fmoc)

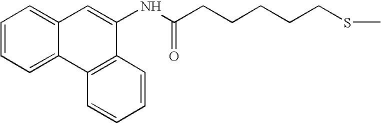

Phenanthroline conjugate

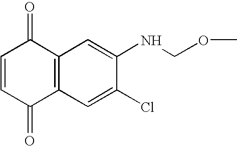

Naphthoquinone conjugate

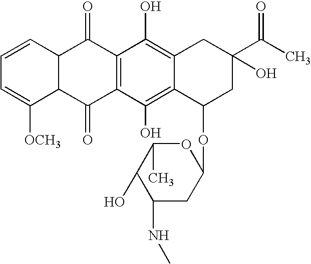

Daunomycin derivatives

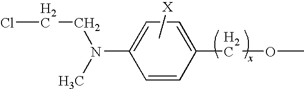

x=1-18, X=alkyl, halogen, NO₂, CN or

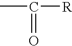

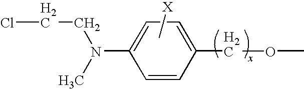

x=1-18, X=alkyl, halogen, NO₂, CN or

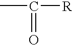

Nanoparticles Attached to Heterocycles Participating in i-motif Formation

Nanoparticles attached to the bioconjugate and/or to the nucleic acid binding compound include but are not limited to metal nanoparticles, e.g., gold, silver, copper and platinum, semiconductor nanoparticles, e.g., CdSe, and CdS, or CdSc coated with ZnS, and magnetic nanoparticles, e.g., ferromagnetic. Other nanoparticles which can be used for the invention include, but are not limited to ZnS, ZnO, TiO₂, Agl, AgBr, HgI₂, PbS, PbSe, ZnTe, CdTe, In₂S₃, In₂Se₃, Cd₃P₂, Cd₃As₂, InAs, and GaAs. The size of the nanoparticles is preferably from about 3 nm to about 250 nm (mean diameter), most preferably from about 5 to about 50 nm. Also, nanoparticles made of latex, plastics, silica, quartz (wafer), glass, zeolithe or any organic material are included in this invention. Additionally, nanoparticles coated with any organic or inorganic material are included. Rods and carbon nanotubes and other nanotubes may also be considered as nanoparticles.

Methods for the preparations of the above mentioned nanoparticles are known to man skilled in the art and have been reported in literature.

In one embodiment of the invention, the bioconjugate and/or the nucleic acid binding compound is attached to a gold nanoparticle. Colloidal gold nanoparticles have high extinction coefficients for the bands that are visible by the eye. These intense colours depend on particle size, concentration, interparticle distance, state of aggregation and geometry of the aggregates. These properties make gold nanoparticles particularly attractive for colorimetric assays.

Backbone within the i-motif

The most popular backbone is the naturally occurring sugar phosphate backbone of nucleic acids containing either ribonucleoside subunits (RNA), deoxyribonucleoside subunits (DNA), peptide nucleic acid subunits (PNA), acyclic subunits or oligosaccharide subunits. Therefore, in a preferred embodiment, the backbone comprises phosphodiester linkages and ribose. In recent years, there have been reports of nucleic acid binding compounds that have similar properties to oligonucleotides, but differ in the structure of their backbone, which have structures formed from any and all means of chemically linking nucleotides, e.g., hexopyranose, 3-deoxy-erythro-pentofuranosyl moiety, as an alternative to the natural occurring phosphodiester ribose backbone.

In a further preferred embodiment, the sugar configuration is selected from the group consisting of the α-D-, β-D-, α-L- and β-L-configurations, most preferably the bioconjugate and/or the nucleic acid binding compound contains at least one 2-deoxy-β-D-erythro-pentofuranosyl moiety or one β-D-ribofuranosyl moiety. In a preferred embodiment of the invention, the backbone is the glycoside C-1 of a sugar moiety of the bioconjugate and/or the nucleic acid binding compound according to the invention. The backbone may include phosphorothioates, methyl phosphonates, phosphoramidates and phosphortriesters linkages. The modifications in the backbone may vary the properties of the bioconjugate and/or the nucleic acid binding compound, i.e., it may enhance stability and affinity.

Therefore, in a preferred embodiment, the bioconjugate and/or the nucleic acid binding compound are those bioconjugates and/or nucleic acid binding compounds in which the backbone comprises one or more moieties of the general Formula 6, but the bioconjugates and/or nucleic acid binding compound are not limited thereto.

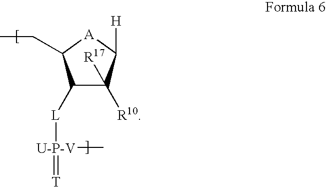

wherein
A is selected from the group consisting of O, S, Se, Te, CH2, N—CO—(C1-C50)-alkyl,
L is selected from the group consisting of oxy, sulfanediyl, —CH2— and —NR11—,
T is selected from the group consisting of oxo, thioxo and selenoxo, telluroxo,
U is selected from the group consisting of —OH, O—, —O-reporter group, —SH, —S, reporter group, —SeH, —(C1-C50)-alkoxy, —(C1-C50)-alkyl, —(C6-C50)-aryl, —(C6-C50)-aryl-(C1-C50)-alkyl, —NR12R13, and —(—O—(C1-C50)-alkyl-)n-R14, wherein n can be any integer between 1 and 6, or wherein —NR12R13 can together with N be a 5-6-membered heterocyclic ring,
V is selected from the group consisting of oxy, sulfanediyl, —CH2-, or —NR11-,
R10 and R17 are independently selected from the group consisting of —H, —OH, —(C1-C50)-alkyl, —(C1-C50)-alkenyl, —(C1-C50)-alkynyl, —(C1-050)-alkoxy, —(C2-C50)-alkenyloxy, —(C2-C50)-alkynyloxy, -halogen, -azido, —O-alkyl, —O-allyl, and —NH2,
R11 is independently selected from the group of —H and —(C1-C50)-alkyl,
R12 and R13 are independently selected from the group consisting of —(C1-C50)-alkyl, —(C1-C50)-aryl, —(C6-C50)-aryl-(C1-C50)-alkyl, —(C1-C50)-alkyl-[NH(CH2)c]d-NR15R16 and a reporter group,
R14 is selected from the group consisting of —H, —OH, -halogen, -amino, —(C1-C50)-alkylamino, —COOH, —CONH2 and —COO(C1-C50)-alkyl and a reporter group,
R15 and R16 are independently selected from the group consisting from —H, —(C1-C50)-alkyl, and —(C1-C50)-alkoxy-(C1-C50)-alkyl and a reporter group,
H is a heterocycle showing the donor/acceptor pattern of cytosine. Examples of the heterocycle are given in the formulae 1-5 (above).

Preferably, in compounds of formula 6, R¹⁰ is hydrogen. Preferred definition of L is oxy. Preferred definition of U is —OH and —O-reporter group. Preferred definition of V is oxy.

Compounds of Formula 6 are especially suited to contain heterocyclic moiety of the invention as an integrated part of the bioconjugate and/or nucleic acid binding compound.

In a further preferred embodiment, the sugar configuration is selected from the group consisting of the α-D-, β-D-, α-L- and β-L-configurations, most preferred the bioconjugate and/or nucleic acid binding compound contains at least one 2′-deoxy-3-D-erythro-pentofuranosyl moiety or one β-D-ribofuranosyl moiety. In a preferred embodiment of the invention, B is the glycoside C-1 of a sugar moiety of the compound according to the invention.

In another embodiment of the invention the sugar is in a locked conformation. LNA (Locked Nucleic Acid) is a class of nucleic acid analogue. LNA oligomers that obey the Watson-Crick base pairing rules and hybridize to complementary oligonucleotides. However, when compared to DNA and other nucleic acid derivatives, LNA provides vastly improved hybridization performance. LNA/DNA or LNA/RNA duplexes are much more thermally stable than the similar duplexes formed by DNA or RNA. In fact, LNA has the highest affinity towards complementary DNA and RNA ever to be reported. In general, the thermal stability of a LNA/DNA duplex is increased 3° C. to 8° C. per modified base in the oligomer.

Within the fields of general molecular biology and molecular diagnostics, five major fields for the application of LNA have been identified which are capture probes, sample preparation, detection of SNP's (Single Nucleotide Polymorphisms), allele specific PCR, and hybridization probes, Molecular Beacons, Padlock probes, Taqman probes (see, for example, international patent application WO92/02638 and corresponding U.S. Pat. No. 5,210,015, U.S. Pat. No. 5,804,375, U.S. Pat. No. 5,487,972) and probes for in-situ hybridizations.

In most respects, LNA may be handled like DNA. LNA is at least as stable as DNA and is soluble in aqueous buffers. LNA can be ethanol precipitated, dried and resuspended, and can be analyzed on gels, HPLC and MALDI-TOF.

LNAs are nucleic acid analogs that can dramatically increase the performance of not only diagnostic assays that probe and evaluate genetic information but also of antisense and other genetic medicine approaches. These analogs, which can be utilized in most applications just like their natural counterparts, lock the nucleic acid into the most productive conformation for hybridization. Hybridization, or complementary docking of genetic probes, is the predominant form of evaluation of genetic information in diagnostics. A broad variety of applications for LNAs have been developed including a number of extremely sensitive and specific assays able to detect specific disease-causing single base mutations in an individual's genes, in the detection of SNPs (Single Nucleotide Polymorphisms), which are the small variations in our genes, that may cause a predisposition to disease, there are data to show that LNA capture probes of only eight nucleotides in length are able to more effectively discriminate between mutated and wild type genes in a sample than much longer conventional nucleic acid capture probes.

Therefore the invention also contemplates bioconjugate and/or nucleic acid binding compounds according to the invention wherein at least one carbon atom of the sugar moiety is connected to at least one other carbon atom of the sugar moiety via at least one bridging moiety containing at least one atom whereby a conformationally constrained sugar is formed as outlined above. Thereby, the sugar is fixed in a locked conformation.

Protecting Groups within the i-motif Structure

The heterocycles according to formulae 1-5 and the backbone are protected with the common protecting groups used in oligonucleotide, peptide or oligosaccharide chemistry and are well known to man skilled in the art or can be selected from publications related to this field or from special reviews or books (see also “Protecting Groups”, edited by P. J. kocieński, Georg Thieme Verlag Stuttgart, 2005).

Residues Linked to the i-motif Structure

The invention concerns composition consisting of an i-motif or an i-motif related structure of the formula 7 and comprise at least one nanoparticle. The i-motif structure or i-motif related structure is formed by at least one bioconjugate (nucleic acid binding compound attached to a nanoparticle) and (i) without or (ii) with at least one further nucleic acid binding compound.

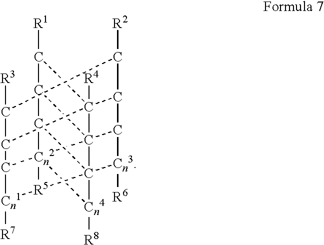

wherein

— represents a connector of any backbone within the i-motif

C represents cytosine residues or derivatives thereof according to formulae 1-5

R₁-R₈ are independently from each other with the proviso that at least one of these residues R₁-R₈ is a nanoparticle and the remaining residues are selected from the group consisting of
(1) any naturally occurring or artificial backbone connected to the i-motif,
(2) oligonucleotides including modified oligonucleotides,
(3) DNA, RNA, LNA, PNA in which one or more sugar moieties exhibit the α-D-, β-D, α-L- and/or β-L-configuration,
(4) heterocycle residues of any structure,
(5) nanoparticle,
(6) microparticle and/or any larger particle,
(7) protecting group,
(8) surface,
(9) reporter group,
(10) linker and connector unit,
(11) dendrimeric structure,
(12) stiff linkers, e.g., formed by incorporation of triple bonds,
(13) multi-linker units,
(14) spacer unit,
(15) linker unit connecting at least two strands of the i-motif with each other forming hairpin structures,
(16) attachment unit,
(17) antibody,
(18) antigenic group,
(19) linker, spacer and/or reporter units with the capability to generate non-covalent interactions (e.g., the biotin-avidin system, antigen-antibody interaction),
(20) delivery unit (e.g., steroids, liposomes),
(21) linker, spacer and/or reporter unit with the capability to form covalent interactions via the Huisgen-Sharpless cycloaddition “click-chemistry”,
(22) —H, and

n¹-n⁴ are independently from each other and are integers between 0 and n.

In addition, all embodiments concerning the modifications (reporter groups, nanoparticle, protecting groups and backbone) within the i-motif structure or i-motif related structure are also disclosed for the residues R¹-R⁸ linked to the i-motif or i-motif related structure.

In another embodiment of the invention, the composition is immobilised on the surface of a substrate via the bioconjugate or the nucleic acid binding compound, including but not limited to a glass substrate, metal surfaces or semiconducting substrates, such as silicon. Further suitable surfaces include surfaces such as glass, quartz, plastics, or other organic or inorganic polymers, surfaces such as white solid surfaces, e.g., TLC silica plates, filter paper, glass fibre filters, cellulose nitrate membranes, nylon membras, and conducting solid surfaces such as indium-tin-oxide. The substrate can be any shape, colour or thickness, but a preferred surface of a substrate will be flat and thin, colourless or opaque.

In a further embodiment of the invention the composition is stabilized by a stabilizer. Said stabilizer comprises modified heterocycles, modified backbones, drugs or dyes, e.g., actinomycin or ethidiumbromide.

Applications

The invention includes numerous applications based on the i-motif DNA-assembly.

Nanomachines

In a recent approach the i-motif structure has been used to design a molecular nanomachine that is driven by pH changes using a quenched and a non-quenched state of a dye induced by the addition of a single-stranded dG-rich oligonucleotide (see D. Liu, S. Balasubramanian, Angew. Chem. Int. Ed. 2003, 42, 5734). The composition described in this invention represents a proton fuelled nanomachine that requires only an i-motif nanoparticle-oligonucleotide conjugate, acid and base but no other additional molecule.

Liedl and Simmel “Switching the Conformation of a DNA molecule with a Chemical Oscillator”, Nano Letters, 2005, vol 5, no 10, 1894-1898, have reported the use of the conformation changes of a cytosine-rich DNA strand between a random coil conformation and an i-motif structure and its possible use as a molecular device. However, the DNA strand used in this work was attached to a dye (Alexa Fluor 488) or a quencher BHQ-1) to allow detection of the conformational changes.

In an embodiment of this invention the composition acts as a nanomachine. Nucleic acid binding compounds which are able to form an i-motif or related structure carrying a nanoparticle (bioconjugate) can be used as pH-sensitive nanoscopic devices. The i-motif structure acts as a pH-dependent switch causing a reversible assembly of the nanoparticles at acidic pH and a disassembling into a disperse nanoparticle solution under alkaline conditions.

pH-Sensitive Colorimetric Sensor

In a further embodiment of this invention the bioconjugate, preferably carrying a gold nanoparticle, can be used as a pH-sensitive colorimetric sensor. The described composition comprises the option to be used as a colorimetric sensor.

Diagnostic of Tumours

In another embodiment of the invention the composition can be used for the detection of tumour cells. The acid induced assembly of the i-motif structure or a related structure formed by bioconjugate makes the system also applicable for tumour cell diagnostic. J. R. Griffiths, Br. J. Cancer 1991, 64, 425 has noted that tumour cells often produce an acidic environment. As the replacement of cytidine residues by analogues thereof produce bioconjugates that react specifically on a defined pH range, i-motif formation occurs selectively in the more acidic medium of the tumour cells but not in the non-mutated cells. The cell encloses the bioconjugate which carries a reporter group. As a result bioconjugate carrying the reporter group is released into the interior of the cell. Thus, the aggregates in the tumour cells can be detected employing methods with respect to the nature of the particular reporter group, e.g., detection of metal nanoparticles by x-rays.

Treatment of Tumours

In another embodiment of the invention the composition can be used for the treatment of tumours. The acid induced assembly of the i-motif structure or a related structure formed by the bioconjugate makes the system also applicable for tumour cell therapy. J. R. Griffiths, Br. J. Cancer 1991, 64, 425 has noted that tumour cells often produce an acidic environment. As the replacement of cytidine residues by analogues thereof produce bioconjugates that react specifically on a defined pH range, i-motif formation occurs selectively in the more acidic medium of the tumour but not in the non-mutated tissue. The bioconjugate is conjugated to a metal nanoparticle preferably to a gold nanoparticle or a magnetite nanoparticle. The bioconjugate is injected into the tumour regions and precipitates, thus forming the composition, preferably in regions in which the cell tissue is more acidic than the healthy tissue. Thus, a tissue selective deposition is possible. The body is subsequently irradiated, e.g., by applying a magnetic field or x-rays, which increases the temperature (hyperthermie) of the tissue marked by the i-motif assemblies of the bioconjugates. This results to a complete or partial destruction of the tumour tissue.

In another preferred embodiment of the invention the composition can be used for the treatment of tumours on the basis of hyperthermy. The tumour tissue is selectively heated in order to initiate the glycolysis metabolism in the cells to start anaerobic lactic acid production. In this local area, the tissue becomes significantly more acidic than other tissues under aerobic respiratory conditions. The bioconjugate is injected into this acidic tissue region. Due to the acidic conditions the composition is formed. The body is subsequently irradiated, e.g., by applying a magnetic field or x-rays, which increases the temperature (hyperthermie) of the tissue marked by the i-motif assemblies of the bioconjugates. This results to a complete or partial destruction of the tumour tissue.

Delivery of Drugs to Cells

In another preferred embodiment of the invention the composition can be used for the release of drugs inside an acidic tumour cell. As the replacement of cytidine residues by analogues thereof produce bioconjugates that react specifically on a defined pH range, i-motif formation occurs selectively in the more acidic medium of the tumour cells but not in the non-mutated cells. A drug is conjugated to the bioconjugate via an acidic labile linker group. The cell encloses the bioconjugate. As a result the bioconjugate carrying the drug is released into the interior of the cell. Under the conditions mentioned above the drug is delivered into the interior of the cell. For example, this drug can bean oligonucleotide designed for the antigen or antisense approach.

Selective Capturing of Samples

The bioconjugate according to the present invention may be used as a capture probe for the determination of the presence, absence or amount of a sample. Capturing will occur via formation of the i-motif structures or the i-motif related structures, thus forming the composition. The bioconjugate should preferably contain a detectable reporter group. The composition formed by the bioconjugate and the sample can then be determined by the detectable reporter group. The release of the sample can be achieved by disassembly of the i-motif structures or i-motif related structures by pH-changes or temperature-changes, as discussed above. This type of assays can be divided into two groups, (i) homogenous assays and (ii) heterogeneous assays.

In heterogeneous assays, preferably the composition will be determined when bound to a solid phase. This embodiment has the advantage that any excess of undesired components can be removed easily from the composition, thus making the determination easier. The composition can be captured to a solid phase either covalently, noncovalently, specifically or unspecifically.

In homogenous assays the composition will not be bound to a solid phase, but will be determined either directly or indirectly in solution, e.g., the bioconjugate allows the capturing of dC-rich oligonucleotides from an oligonucleotide library under acidic conditions in solution, thus forming the composition.

In another preferred embodiment, the invention further provides kits for the detection of the presence, absence or amount of any sample. In one embodiment the kit comprises at least a container holding the bioconjugate in an aggregated (composition) or non-aggregated state. The bioconjugate is capable of forming i-motif structures or i-motif related structures with the sample.

Deposition of Metal Nanoparticles on a Surface (Nano-Arrays)

In another preferred embodiment the nucleic acid binding compound is conjugated to at least one metal nanoparticle, preferably a gold nanoparticle, to form the bioconjugate.

Metal nanoparticles, preferably gold can be deposited onto the surface of a substrate (nano-array) in which regions of this array are made acidic (for example, by etching with HF). The gold nanoparticles are attached to the bioconjugates which form an i-motif or an i-motif related structure at a particular pH value and then aggregate to form the composition at the regions having this pH value. This could be used, for example, for the deposition of extremely thin conducting strips on an insulating substrate or to add bar codes to a product.

One example according to the method mentioned above includes the construction of nanoparticle patterns on a surface. On a solid organic or inorganic material, e.g., a glass plate, acid or an acidic compound will be deposited at defined sites on the surface creating a defined pattern. The surface will be soaked entirely with a solution containing the bioconjugate conjugated to a metal nanoparticle, preferable a gold nanoparticle. The bioconjugate will assemble to an i-motif structure only at positions where the surface is acidic but at not at other places. The excess of the solution containing the bioconjugate will be washed off while the assembled particles stay on the surface. If the bioconjugates containing gold nanoparticles are used a gold pattern is created. Depending on the application the nucleic acid binding compound can be kept in the assembly or removed. This will result in a pattern of gold molecules which are forming wires which can be used as electronic circuits.

AFM Detection

In another embodiment the composition is conjugated to at least one nanoparticle that is directly detectable by atomic force microscopy (AFM). This offers the opportunity to calculate the size of a given nanoparticle and to determine its location on a surface of the substrate.

SEM, TEM and Related Techniques

In another embodiment the composition is conjugated to at least one nanoparticle that is directly detectable by scanning electron microscopy, tunnel electron microscopy and related techniques.

Catalysis

In another preferred embodiment the composition is conjugated to at least one nanoparticle that shows catalytic activity. In acidic solution the composition conjugated to catalytic active nanoparticles prevents the unspecific aggregation of the nanoparticles. Thus, the highest catalytic activity is provided to the system.

Nanoparticles showing catalytic activity can be deposited onto the surface of a substrate (nano-array) in which regions of this array are made acidic. This allows the highest distribution of the nanoparticles at defined sites of the surfaces and prevents an unspecific aggregation of these nanoparticles. Therefore it is possible to remove any pollutant from any liquid or gas phase.

In another preferred embodiment the composition is conjugated to a nanoparticle and to an enzyme via the heterocycle or any residue R₁-R₄ (Formula 7). An enzyme catalyzed reaction can be performed in solution by adding the composition. As the replacement of cytidine residues by analogues thereof produce nucleic acid binding compounds that react specifically on a defined pH range, i-motif formation can occurs selectively in a pH-range at which the enzyme shows no activity. pH-Changes of the solution either activates the enzyme or removes the enzyme from the solution by i-motif formation or disassembling of the i-motif. This allows the possibility to switch on and to switch off the enzyme.

### Specific embodiments

The present invention is explained in more detail by the following examples:

### Example 1

### Synthesis, Purification, and Characterization of Oligonucleotides (Nucleic Acid Binding Compound)

### 1.1 Chemicals and Instrumentation

HAuCl4.3H₂O and trisodium citrate were purchased from Aldrich (Sigma-Aldrich Chemie GmbH, Deisenhofen, Germany). The 5′-sulfanyl modifier 6-[(triphenylmethyl)sulfanyl]hexyl(2-cyanoethyl) N,N-diisoproylphosphoramidite was obtained from Glen Research (Virginia, USA). UV/VIS spectra were recorded with a U-3200 spectrophotometer (Hitachi, Tokyo, Japan); λₘₐₓ (ε) in nm. CD-spectra were measured as accumulations of three scans with a Jasco 600 (Jasco, Japan) spectropolarimeter with thermostatically (Lauda RCS-6 bath) controlled 1-cm quartz cuvette.

### 1.2 Preparation of Gold Nanoparticles

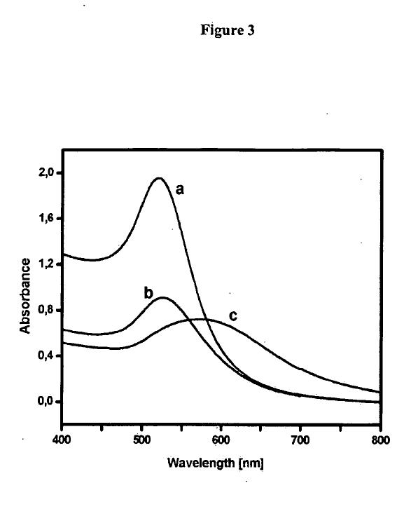

The 15 nm gold nanoparticle solutions were prepared from a HAuCl₄ solution by citrate reduction as it was originally reported in Turkevitch, P. C. Stevenson, J. Hillier, Discuss. Faraday Soc. 1951, 11, 55 and later described by Letsinger and Mirkin (J. J. Storhoff, R. Elghanian, R. C. Mucic, C. A. Mirkin, R. L. Letsinger, J. Am. Chem. Soc. 1998, 120, 1959 and R. Jin, G. Wu, Z. Li, C. A. Mirkin, G. C. Schatz, J. Am. Chem. Soc. 2003, 125, 1643). All glassware was cleaned in aqua regia (3 parts HCl, 1 part HNO₃), rinsed with nanopure water, then oven dried before use. Aqueous HAuCl₄ (1 mM, 250 ml) was brought to reflux while stirring. Then, 38.8 mM tri-sodium citrate (25 ml) was added quickly. The solution colour changed from yellow to red, and refluxing was continued for 15 min. After cooling to room temperature, the red solution was filtered through a Micron Separations Inc. 1 micron filter. Prior to functionalization the gold nanoparticle solution was brought up from pH 5.5 to pH=9.5 to avoid i-motif formation of the non-derivatized oligonucleotides. The UV/VIS spectrum of the alkaline solution of the unmodified nanoparticles shows the characteristic plasmon resonance at 520 nm appearing at the same wavelength as observed for the acidic solution (FIG. 3, spectrum a).

### 1.3 Synthesis of Oligonucleotides

Four different oligonucleotides, namely 5′-d(TTC CCC TT) (1) and 5′-d(TTC CCC CCT T) (2) and their 5′-thiol-modified derivatives 3 and 4 were prepared.

### 1.3a Synthesis of the Unmodified Oligonucleotides

The unmodified oligonucleotides were synthesized by solid phase synthesis on 1 μmol scale using a DNA synthesizer (ABI 392-08, Applied Biosystems, Weiterstadt, Germany) employing phosphoramidite chemistry [Users' Manual of the DNA synthesizer, Applied Biosystems, Weiterstadt, Germany, p. 392]. The dimethoxytrityl (DMT) protecting group was not cleaved from the oligonucleotides to aid in purification. The oligonucleotides were deprotected with 25% aq. NH₃ (60° C., 16 h).

### 1.3b Synthesis of the 5′-thiol-Modified Oligonucleotides

The syntheses of the 5′-thiol modified oligonucleotides were performed as described for the unmodified oligonucleotides employing a 5′-thiol-modifier C6-phosphoramidite reagent (Glen Research, US). Deprotection of the 5′-thiol modified oligonucleotides was performed with 25% aq. NH₃ (60° C., 16 h).

### 1.4 Purification of Oligonucleotides

### 1.4a Purification of the Unmodified Oligonucleotides

Purification of the unmodified 5′-dimethoxytrityl oligomers was performed by reversed-phase HPLC (RP-18) in the trityl-on modus with the following solvent gradient system [A: 0.1 M (Et₃NH)OAc (pH 7.0)/MeCN 95:5; B: MeCN]: 3 min, 20% B in A, 12 min, 20-50% 13 in A and 2-5 min, 20% B in A with a flow rate of 1.0 ml/min. The solution was dried and treated with 2.5% CHCl₂COOH/CH₂Cl₂ for 5 min at 0° C. to remove the 4,4′-dimethoxytrityl residues. The detritylated oligomers were purified by reverse phase HPLC with the gradient: 0-20 min, 0-20% B in A with a flow rate of 1.0 ml/min. The oligomers were desalted (RP-18, silica gel) and lyophilized in a speed-Vac evaporator to yield colourless solids which were frozen at −24° C.

### 1.4b Purification of the 5′-thiol-modified Oligonucleotides

The trityl-protected oligonucleotides 3 and 4 were purified by reverse phase HPLC in the trityl-on modus as described for the unmodified oligonucleotides.

The trityl-protecting groups of 3 and 4 were removed immediately before modification with the gold nanoparticles. The trityl-protecting group was cleaved by treatment of the dry oligonucleotide samples with 150 μl of a 50 mM AgNO₃ solution. A milky suspension was formed which was allowed to stand for 20 min at room temperature. Then, 200 μl of a 10 μg/ml solution of dithiothreitol (5 min) were added. A yellow precipitate was formed which was removed by centrifugation (30 min, 14000 rpm) [see J. J. Storhoff, R. Elghanian, R. C. Mucic, C. A. Mirkin, R. L. Letsinger, J. Am. Chem. Soc. 1998, 120, 1959]. Aliquots of the samples were purified on a NAP-10 column (Sephadex G-25 Medium, DNA grade; Amcrsham Bioscience AB, S-Uppsala; equilibrated with 15 ml of nanopure water).

### 1.5 Characterization of the Oligonucleotides 1-4 by MALDI-TOF Spectroscopy

The molecular masses of the oligonucleotides were determined by MALDI-TOF with a Biflex-III instrument (Bruker Saxonia, Leipzig, Germany) and 3-hydroxypicolinic acid (3-HPA) as a matrix (Bruker Saxonia, Leipzig, Germany). The oligonucleotides 1 and 2 were characterized after complete deprotection and HPLC purification, followed by desalting. Oligonucleotides 3 and 4 were characterized after HPLC purification on the trityl-on level.

In all cases, the calculated masses were in good agreement with the measured values (Table 1).

TABLE 1
Molecular masses of oligonucleotedes determined by MALDI-TOF mass spectrometry.

| | [M + H]⁺ [Da] | [M + H]⁺ [Da] |
|---|---|---|
| Oligonucleotides | (calc.) | (found) |
| 5′-d(T-T-C-C-C-C-T-T) (1) | 2312 | 2311 |
| 5′-d(T-T-C-C-C-C-C-C-T-T) (2) | 2890 | 2891 |
| 5′-Trityl-S—(CH₂)₆—O(PO₂H)O-d(T-T-C-C-C-C-T-T) (3) | 2751 | 2750 |
| 5′-Trityl-S—(CH₂)₆—O(PO₂H)O-d(T-T-C-C-C-C-C-C-T-T) (4) | 3329 | 3328 |

### Example 2

### Synthesis of Modified Gold Nanoparticle (Bioconjugate) and their Characterization

### 2.1 Synthesis of Modified Gold Nanoparticles

The trityl-protecting groups of 3 and 4 were removed immediately before modification with the gold nanoparticles. The trityl-protecting group was cleaved by treatment of the dry oligonucleotide samples with 150 μl of a 50 mM AgNO₃ solution. A milky suspension was formed which was allowed to stand for 20 min at room temperature. Then, 200 μl of a 10 μg/ml solution of dithiothreitol (5 min) were added. A yellow precipitate was formed which was removed by centrifugation (30 min, 14000 rpm) [see J. J. Storhoff, R. Elghanian, R. C. Mucic, C. A. Mirkin, R. L. Letsinger, J. Am. Chem. Soc. 1998, 120, 1959]. Aliquots of the samples were purified on a NAP-10 column (Sephudex G-25 Medium, DNA grade; Amersham Bioscience AB, S-Uppsala; equilibrated with 15 ml of nanopure water). The effluents from 0 to 2.5 ml were collected and the volume was adjusted to 3.5 ml with nanopure H₂O.

Prior to modification the gold nanoparticle solution was brought to pH 9.5. The oligonucleotide modified gold nanoparticles were synthesized by derivatizing 6.4 ml of the alkaline gold nanoparticle solution with 3.5 ml of the 5′-(sulfanylalkanyl)-modified oligonucleotide solution. The solution was allowed to stand for 20 h at 40° C. followed by the addition of 4.8 ml of a 0.1 M NaCl, 10 mM phosphate buffer solution (pH 7). The solution was kept for further 2 days at 40° C. The sample was centrifuged using screw cap micro tubes for 30 min at 14000 rpm. The clear supernatant was removed and the red oily precipitate was washed two times with 8.4 ml of 0.1 M NaCl, 10 mM phosphate buffer solution (pH 7) and redispersed in 9.6 ml of a 0.1 M NaCl or 0.3 M NaCl, 10 mM phosphate buffer solution (pH 8.5).

The DNA modified gold nanoparticles (bioconjugate) 5 and 6 are stable in sodium phosphate buffer containing 0.1 or 0.3 M NaCl at pH=8. No detectable aggregation was observed after 1-2 months as evidenced by UV/VIS spectroscopy.

### 2.2 Characterization of the Bioconjugates

The resulting gold-DNA bioconjugates show the expected plasmon resonance at 525 nm under alkaline conditions indicating a non-aggregated state (FIG. 3, spectrum b).

### Example 3

### Formation of the i-motifs and their Characterization

### 3.1 Formation of the i-motif

The i-motif was stabilized by hemiprotonated non-canonical cytosine-cytosine base pairs in which a protonated dC⁺ is situated opposite to an unprotonated dC residue. Due to the partly required protonation of the cytosine residues the i-motif assembly was formed under slightly acidic conditions (pH 5.5).

### 3.2 Characterization of the i-motif

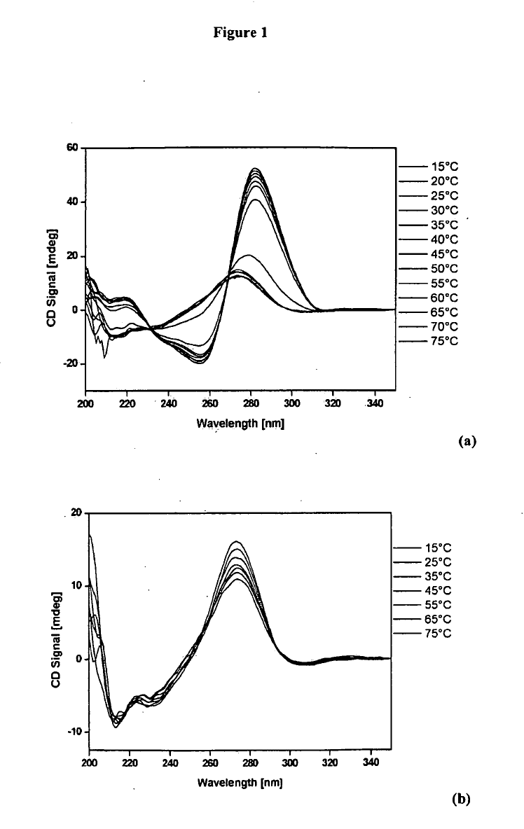

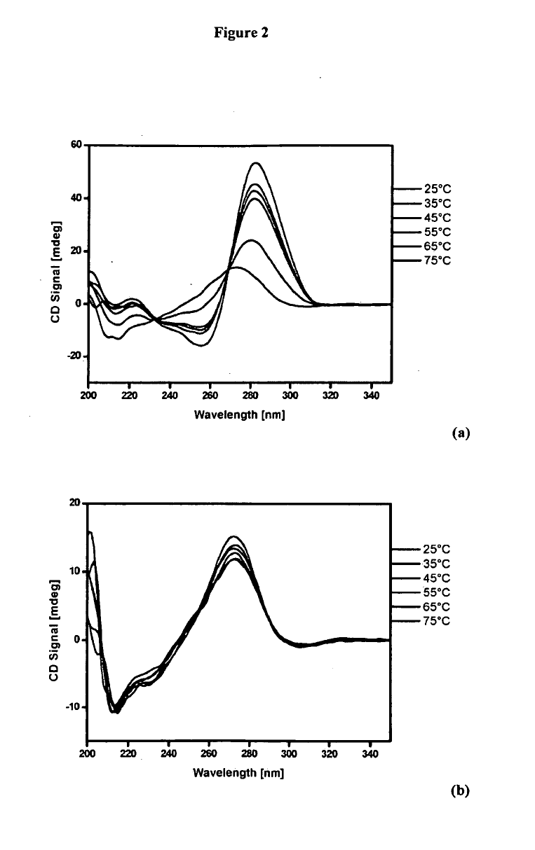

The distinct characteristics of the i-motif can be monitored by circular dichroism (CD) spectra. In the aggregated state a positive band around 280 nm and a concomitant negative band around 260 nm are typical for the i-motif structure. The bands appear under slightly acidic conditions and change in alkaline medium (G. Manzini, N. Yathindra, L. E. Xodo, Nucleic Acids Res, 1994, 22, 4634 and F. Seela, Y. He, in ‘Organic and Bioorganic Chemistry’, Ed. D. Loakes, Transworld Research Network, 2002, p. 57). The formation of the i-motif structure of the unmodified oligonucleotides 1 (FIGS. 1) and 2 as well as the modified derivatives 3 and 4 carrying bulky protecting groups was established by CD measurements (FIG. 2). A large positive lobe at 282 nm and a negative lobe at 256 nm (pH=5.2) were indicative for the i-motif structure shown in FIG. 2a. The CD-spectra changed by increasing the temperature (FIG. 2a) or by shifting the pH towards alkaline medium due to the disassembly of the i-motif (FIG. 2b).

### Example 4

### Formation of the Compositions and their Characterization

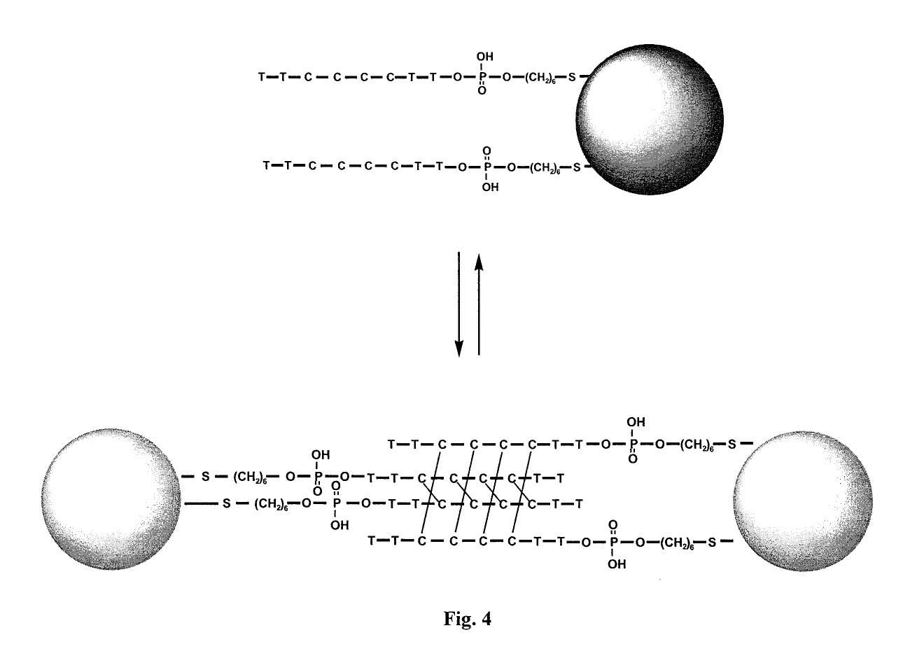

dC-rich DNA forms an i-motif under acidic condition (pH 5.5). The same occurs for the bioconjugate which forms the composition below pH 5.5. The UV/VIS spectrum of the composition shows a shift of the plasmon resonance band from 525 nm to y in comparison to the non-aggregated bioconjugate as indicated by FIG. 3, spectrum c. A disassembly of the composition is observed at higher pH-values (see FIG. 4).

### Example 5

### pH-Dependent Colorimetric Assay Based on the Formation of the Composition

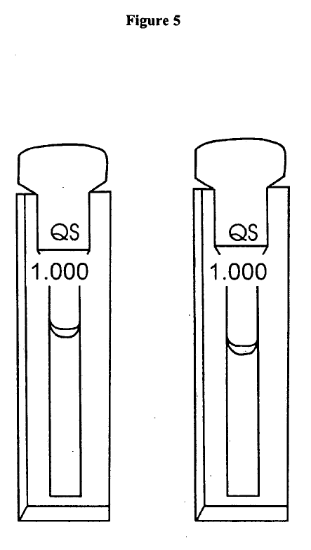

The assembly and disassembly of the composition 5 was accompanied by a dramatic colour change from deep red to blue between pH 6.5 and 5.5 as shown in FIG. 5. The colour change occurred within less a second, was fully reversible and was repeatable. Therefore the composition can be used as a colorimetric sensor.

### Example 6

### Switchable Nanoscaled Devices (Nanomachine) Based on the Formation of the Composition

The response to an external stimulus is a basic requirement of a switchable nanoscaled device.

In a recent approach the i-motif structure has been used to design a molecular nanomachine that is driven by pH changes using a quenched and a non-quenched state of a dye induced by the addition of a single-stranded dG-rich oligonucleotide (D. Liu, S. Balasubramanian, Angew. Chem. Int. Ed. 2003, 42, 5734).

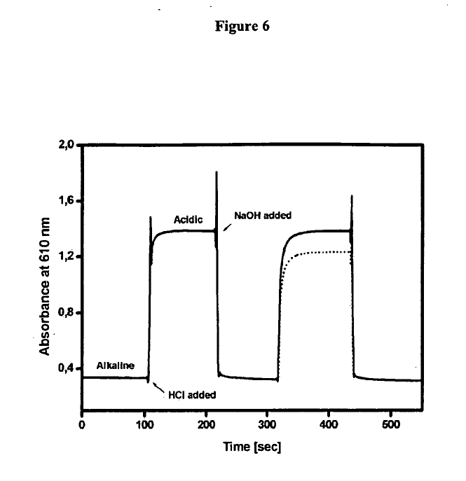

The bioconjugate 5 showed an on-state below pH 5.5 refering to the formation of the composition. A disassembly of the composition (off-state) occurs at higher pH-values. The pH-dependent assembly of the nanoparticles can be examined by acid or base addition to the solution. The reaction can be followed spectrophotometrically. As shown in FIG. 6 the switching between the two states is fully reversible and can be repeated by multiple working cycles. Multiple cyclic additions of HCl and NaOH to the functionalized gold-nanoparticle solution (700 μl; 10 mM phosphate buffer with 0.1 M NaCl) results in changes of the UV-absorbance measured at 610 nm. This confirms the formation of the composition induced by the i-motif.

The composition represents a proton fuelled nanomachine which requires only a bioconjugate, acid and base but no other additional molecule.

### Example 7

### Properties of 5-propynyl-2′-deoxycytidine

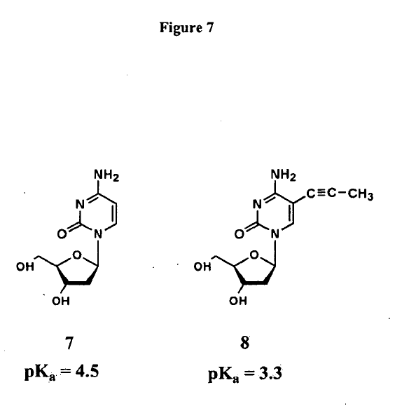

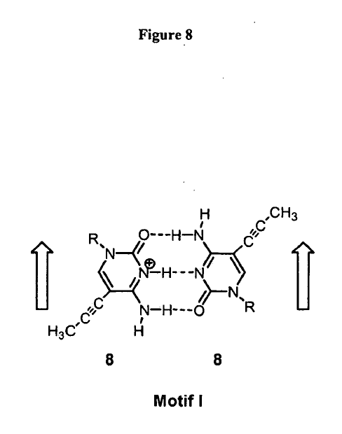

Due to the introduction of the propynyl group at position-CS of the pyrimidine ring the pkₐ value of the heterocycle was lowered from 4.5 to 3.3 (see J. Robles, A. Granadas, E. Pedroso, F. J. Luque, R. Eritja, M. Orozco, Curr. Org. Chem. 2002, 6, 1333) as shown in FIG. 7. As a consequence, oligonucleotides incorporating 5-propynylcytosine (8) residues required lower pH values for the protonation of N-3 which contributes to the formation of the hemiprotonated tridentate dC*•dC(+)* base pair (FIG. 8).

### Example 8

### Synthesis, Purification and Characterization of Modified Oligonucleotides (Nucleic Acid Binding Compounds) Incorporating 5-propynyl-cytosine Residues

### 8.1 Synthesis of the Modified Oligonucleotides Incorporating 5-propenyl-cytosine Residues

The modified oligonucleotides 9-11 incorporating a 5′-thiol-modifier-C6-phosphoramidite (Glen Research, US) and the phosphoramidite of 5-propynyl-2′-deoxycytidine (see F. W. Hobbs, J. Org. Chem. 1989, 54, 3420) were synthesized by solid phase synthesis on 1 μmol scale using a DNA synthesizer (ABI 392-08, Applied Biosystems, Weiterstadt, Germany) employing phosphoramidite chemistry [Users' Manual of the DNA synthesizer, Applied Biosystems, Weiterstadt, Germany, p. 392]. Deprotection of the 5′-thiol modified oligonucleotides was performed with 25% aq. NH₃ (60° C., 16 h). For the 3′-thiol modification the 3′-thiol-modifier-C3 S-S CPG was used (Glen Research, US). Deprotection was performed as described for the 5′-thiol modified oligonucleotides.

### 8.2 Purification of the 3′-thiol-modified Oligonucleotide 9

Purification of the modified oligonucleotide 9 was performed by reversed-phase HPLC (RP-18) in the DMT-on modus with the following solvent gradient system [A: 0.1 M (Et₃NH)OAc (pH 7.0)/MeCN 95:5; B: MeCN]: 3 min, 20% B in A, 12 min, 20-50% B in A and 25 min, 20% B in A with a flow rate of 1.0 ml/min. The solution was dried and treated with 80% CH₃COOH for 30 min at r.t. to remove the 4,4′-dimethoxytrityl residues. The detritylated oligonucleotide was precipitated with 300 μl 1M NaCl solution and 1 ml ethanol under cooling.

### 8.3 Purification of the Modified Oligonucleotides 10 and 11

Purification of the modified oligonucleotides 10 and 11 was performed by reversed-phase HPLC (RP-18) in the trityl-on modus with the following solvent gradient system [A: 0.1 M (Et₃NH)OAc (pH 7.0)/MeCN 95:5; B: MeCN]: 3 min, 20% B in A, 12 min, 20-50% B in A and 25 min, 20% B in A with a flow rate of 1.0 ml/min.

The trityl-protecting groups of 10 and 11 were removed immediately before modification with the gold nanoparticles. The trityl-protecting group was cleaved by treatment of the dry oligonucleotide samples with 15.0 μl of a 50 mM AgNO₃ solution. A milky suspension was formed which was allowed to stand for 20 min at room temperature. Then, 200 μl of a 10 μg/ml solution of dithiothreitol (5 min) were added. A yellow precipitate was formed which was removed by centrifugation (30 min, 14000 rpm) [see J. J. Storhoff, R. Elghanian, R. C. Mucic, C. A. Mirkin, R. L. Letsinger, J. Am. Chem. Soc. 1998, 120, 1959]. Aliquots of the samples were purified on a NAP-10 column (Sephadex G-25 Medium, DNA grade; Amersham Bioscience AB, S-Uppsala; equilibrated with 15 ml of nanopure water).

### 8.4 Characterization of the Oligonucleotides 9-11 by MALDI-TOF Spectroscopy

The molecular masses of the modified oligonucleotides 9-11 were determined by MALDI-TOF with a Biflex-III instrument (Bruker Saxonia, Leipzig, Germany) and 3-hydroxypicolinic acid (3-HPA) as a matrix (Bruker Saxonia, Leipzig, Germany). The molecular mass of oligonucleotide 9 was determined after precipitation. Oligonucleotides 10 and 11 were characterized after HPLC purification on the trityl-on level. In all cases, the calculated masses were in good agreement with the measured values (Table 2).

TABLE 2
Thiol-modified oligonucleotides 9-11 and their molecular
masses determined by MALDI-TOF mass spectrometry.

| | [M + H]⁺ [Da] | |
|---|---|---|
| Oligonucleotides | calc. | found |
| 5′-d(T-T-8-8-8-8-8-8-T-T)-(CH₂)₃—S—S—(CH₂)₃—OH (9) | 3362 | 3363 |
| 5′-d{[Trityl-S—(CH₂)₆—O(PO₂H)O]-T-T-8-8-8-8-T-T} (10) | 2903 | 2903 |
| 5′-d{[Trityl-S—(CH₂)₆—O(PO₂H)O]-T-T-8-8-8-8-8-8-T-T} (11) | 3557 | 3557 |

### Example 9

### Synthesis of Bioconjugates Incorporating 5-propynyl-cytosine Residues and their Characterization

### 9.1 Synthesis of Bioconiugates 12 and 13

The trityl-protecting groups of 10 and 11 were removed immediately before modification with the gold nanoparticles. The trityl-protecting group was cleaved by treatment of the dry oligonucleotide samples with 150 μl of a 50 mM AgNO₃ solution. A milky suspension was formed which was allowed to stand for 20 min at room temperature. Then, 200 μl of a 10 μg/ml solution of dithiothreitol (5 min) were added. A yellow precipitate was formed which was removed by centrifugation (30 min, 14000 rpm) [see J. J. Storhoff, R. Elghanian, R. C. Mucic, C. A. Mirkin, R. L. Letsinger, J. Am. Chem. Soc. 1998, 120, 1959]. Aliquots of the samples were purified on a NAP-10 column (Sephadex G-25 Medium, DNA grade; Amersham Bioscience AB, S-Uppsala; equilibrated with 15 ml of nanopure water). The effluents from 0 to 2.5 ml were collected and the volume was adjusted to 3.5 ml with nanopure H₂O.

Prior to modification the gold nanoparticle solution was brought to pH 9.5. The oligonucleotide modified gold nanoparticics were synthesized by derivatizing 6.4 ml of the alkaline gold nanoparticle solution with 3.5 ml of the 5′-(sulfanylalkanyl)-modified oligonucleotide solution. The solution was allowed to stand for 20 h at 40° C. followed by the addition of 4.8 ml of a 0.1 M NaCl, 10 mM phosphate buffer solution (pH 7). The solution was kept for further 2 days at 40° C. The sample was centrifuged using screw cap micro tubes for 30 min at 14000 rpm. The clear supernatant was removed and the red oily precipitate was washed two times with 8.4 ml of 0.1 M NaCl, 10 mM phosphate buffer solution (pH 7) and redispersed in 9.6 ml of a 0.1 M NaCl or 0.3 M NaCl, 10 mM phosphate buffer solution (pH 8.5).

The DNA modified gold nanoparticles (bioconjugate) 12 and 13 (Table 3) are stable in sodium phosphate buffer containing 0.1 or 0.3 M NaCl at pH=8. No detectable aggregation was observed after 1-2 months as evidenced by UV/VIS spectroscopy.

TABLE 3
Structure of the bioconjugates 12 and 13
Bioconjugates

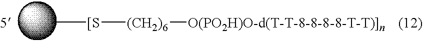

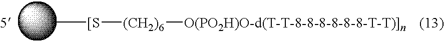

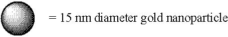

### 9.2 Characterization of the Bioconjugates

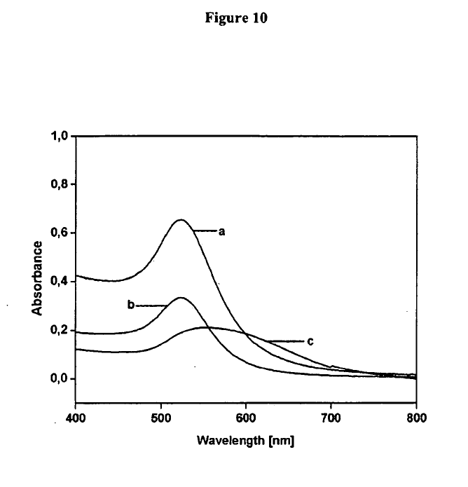

The resulting gold-DNA bioconjugates show the expected plasmon resonance at 525 nm under alkaline conditions indicating a non-aggregated state (FIG. 10, spectrum b).

### Example 10

### Formation of i-motifs Incorporating 5-propynyl-2′-deoxycytidine and their Characterization

### 10.1 Formation of the i-motif Incorporating 5-propenyl-2′-deoxycytidine

The replacement of the cytidine residues by 5-propynyl-2′-deoxycytidine strongly effects the formation of the i-motif structure. Due to the introduction of the propynyl group at position-C5 of the pyrimidine ring the pkₐ value of the heterocycle is lowered from 4.5 to 3.3. As a consequence, oligonucleotides incorporating 5-propynyl-2′-deoxycytidine require lower pH values for the protonation of N-3. Thus, in this case the i-motif is formed at pH=3.3

### 10.2 Characterization of the i-motif

The distinct characteristics of the i-motif were monitored by circular dichroism (CD) spectra according to example 3, 3.2.

It was demonstrated that CD-spectra of cytidine-rich oligonucleotides measured at mild acidic conditions (pH=5.5) show the distinct characteristics of the i-motif structure whereas corresponding CD-measurements of oligonucleotides in which dC is substituted by 8 indicated a non-aggregated state. However, the self-assembly of these oligonucleotides into the i-motif structure is achieved at a pH-value of 3.5.

### Example 11

### Formation of Compositions Incorporating 5-propynyl-2′-deoxycytidine and their Characterization

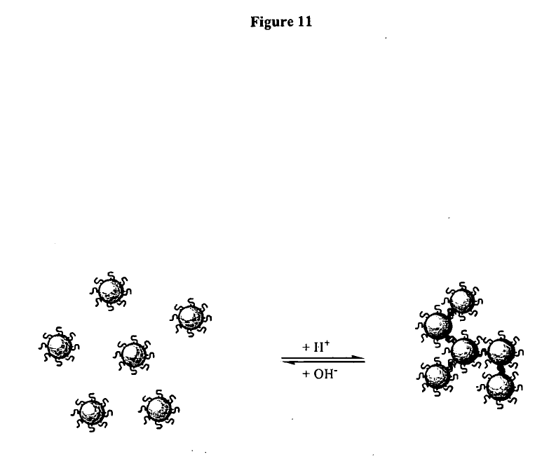

As the formation of the i-motif structure requires acidic conditions the same is valid for the bioconjugates 10 and 11 incorporating 5-propynyl-2′-deoxycytidine. It was shown that at acidic pH values a reversible self-assembly of the bioconjugates forming compositions occured which was indicated by a red shift of the plasmon resonance from 525 nm to 549 nm accompanied by a colour change of the solution from red to blue (FIG. 10, spectrum c). The addition of alkaline phosphate buffer led to the disassembly of the aggregates (FIG. 11).

### Example 12

### Properties of Compositions Incorporating 5-propynyl-2′-deoxycytidine

### 12.1 pH-Dependent Assembly of the Bioconjugates

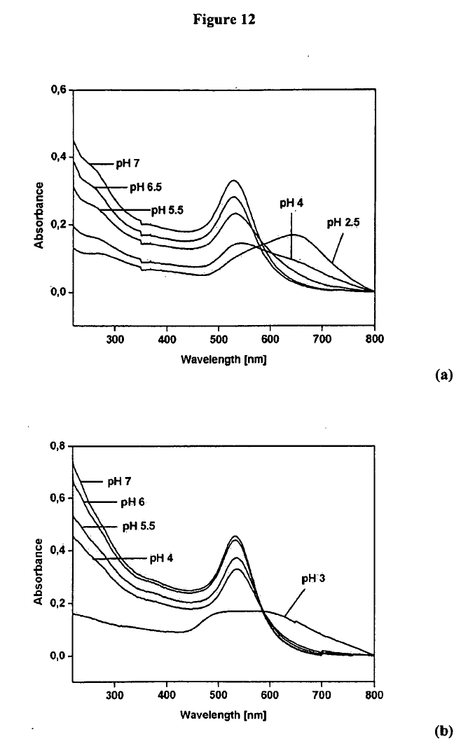

Comparison of compositions containing unmodified dC residues with compositions incorporating 5-propynyl-2′-deoxycytidine by UV/VIS spectroscopy clearly indicates their formation at different pH-values (FIG. 12).

12.2 Kinetics of the Formation of the Compositions

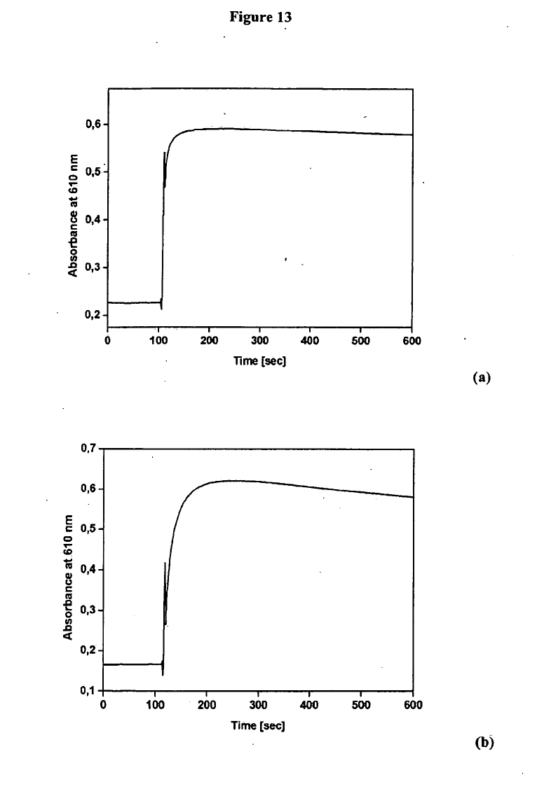

Comparison of the kinetics of bioconjugates 5 and 11 after the addition of 20 μl of 1M HCl show UV/VIS absorption changed indicating the formation of the compositions (FIG. 13). The spectra indicate that the kinetics of the formation of the compositions are very fast.

### Example 13

### Synthesis of 2′-deoxycytidine Analogues

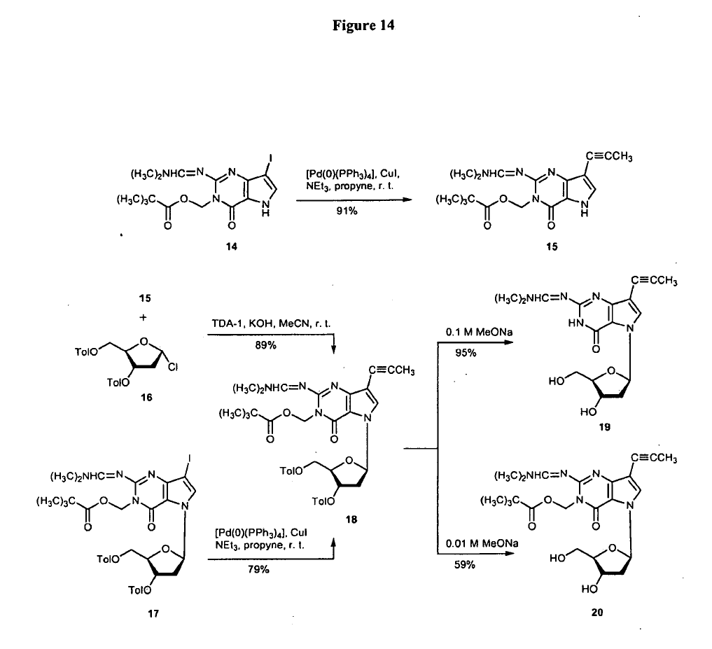

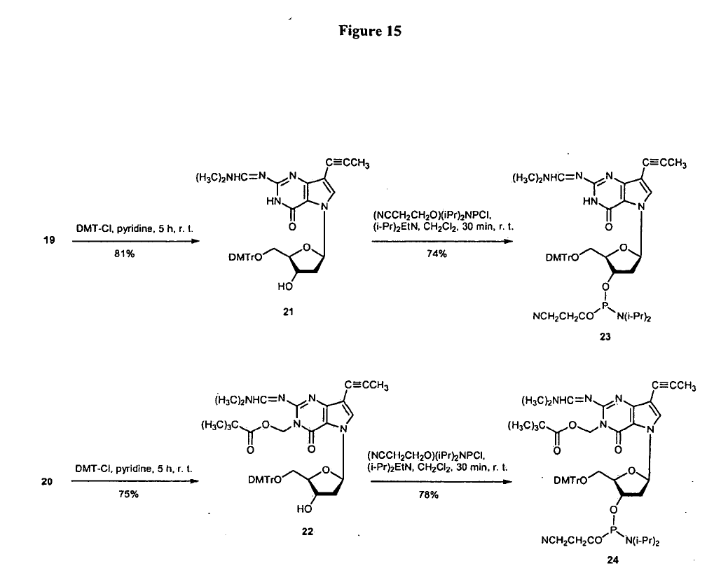

The synthesis of 2′-deoxycytidine analogues resembling the donor/acceptor pattern of 2′deoxycytidine was performed according to FIGS. 14 and 15. This class of analogues does not require protonation for the formation of hemiprotonated base pairs.

### 2-{[(Dimethylamino)methylidene]amino}-3,5-dihydro-3-[(pivaloyloxy)methyl]-7-(prop-1-ynyl)-4H-pyrrolo[3,2-d]pyrimidin-4-one (15)

To the solution of 2-{[(dimethylamino)methylidene]amino}-3,5-dihydro-7-iodo-3-[(pivaloyloxy)methyl]-4H-pyrrolo[3,2-d]pyrimidin-4-one (14) (1.5 g, 3.37 mmol) (see F. Seela, K. I. Shaikh, T. Wiglenda and P. Leonard, Helv. Chim. Acta, 2004, 87, 2507-2516) in anhydrous DMF (10 ml) tetrakis(triphenylphosphine)palladium (0) [(PPh₃)₄Pd(0)] (348 mg, 0.33 mmol), CuI (204 mg, 1.07 mmol) and triethylamine (840 μl, 5.98 mmol) were added while stirring. The sealed suspension was saturated with propyne at 0° C. and stirred at r.t. for 24 h. The solvent was evaporated in vacuo, the reaction mixture dissolved in MeOH (5 ml) and adsorbed on silica gel (4 g). The resulting powder was subjected to FC (silica gel, column 15×3 cm, CH₂Cl₂/MeOH, 95:5). From the main zone compound 15 was isolated as a colorless solid (1.103 mg, 91%) (Found: C, 60.32; H, 6.40; N, 19.43%. C₁₈H₂₃N₅O₃ requires C, 60.49; H, 6.49; N, 19.59); TLC (silica gel, CH₂Cl₂/MeOH, 95:5): R_{f}0.38; λₘₐₓ (MeOH)/nm 223 (ε/dm³ mol⁻¹ cm^{31 1} 26000), 266 (25200) and 308 (16600); δ_{H} (250.13 MHz; [d₆]DMSO; Me₄Si) 1.07 (9H, s, 3 CH₃), 2.01 (3 H, s, CH₃), 2.96, 3.15 (6H, 2s, 2 NCH₃), 6.16 (2H, s, OCH₂), 7.43 (1H, d, J2.97, 8-H), 8.49 (1H, s, N═CH), 12.03 (1H, s, NH).

### 5-[2-Deoxy-3,5-di-O-(p-toluoyl)-β-D-erythro-pentofuranosyl]-{[2-(dimethylamino)methylidene]amino}-3,5-dihydro-3-[(pivaloyloxy)methyl]-7-(prop-1-ynyl)-4H-pyrrolo[3,2-d]pyrimidin-4-one (18)

Method A: To a suspension of powdered KOH (140 mg, 2.50 mmol) and TDA-1 (tris[2-(2-methoxyethoxy)ethyl]amine, 46 μl, 0.14 mmol) in anhyd. MeCN (10 ml) was added compound 15 (725 mg, 2.03 mmol) while stirring at r. t. The stirring was continued for another 10 min and 2-deoxy-3,5-di-O-(p-toluoyl)-α-D-erythro-pentofuranosyl chloride (16) (970 mg, 2.50 mmol) was added in portions. After 30 min insoluble material was filtered off and the solvent was evaporated. The resulting foam was applied to FC (silica gel, column, 12×3 cm, CH₂Cl₂/(CH₃)₂CO, 98:2). A colourless foam 18 was isolated from the main zone (1.28 gm, 89%). Method B: To the solution of compound 17 (850 mg, 1.06 mmol) (see F. Seela, K. I. Shaikh, T. Wiglenda and P. Leonard, Helv. Chim. Acta, 2004, 87, 2507-2516) in anhydrous DMF (6 ml) tetrakis(triphenylphosphine)palladium(0) [(PPh₃)₄Pd(0)] (116 mg, 0.1 mmol), CuI (68 mg, 0.36 mmol) and triethylamine (280 μl, 1.99 mmol) were added. The sealed suspension was saturated with propyne at 0° C. and stirred at r. t. for 24 h. The solvent was evaporated to dryness. The residue was dissolved in MeOH (4 ml), adsorbed on silica gel (2 g) and subjected to FC (silica gel, column 15×3 cm, CH₂Cl₂/(CH₃)₂CO, 98:2). From the main zone compound 18 was isolated as a colorless foam (598 mg, 79%) (Found: C, 65.98;H, 6.03; N, 9.92%. C₃₉H₄₃N₅O₈ requires C, 65.99; H, 6.11; N, 9.87%); TLC (silica gel, CH₂Cl₂/(CH₃)₂CO, 95:5): R_{f}0.75; λₘₐₓ (MeOH)/nm 235 (ε/dm³ mol⁻¹ cm⁻¹ 43100), 266 (23300) and 312 (16600); δ_{H} (250.13 MHz; [d₆]DMSO; Me₄Si) 1.06 (9H, s, 3 CH₃), 2.01 (3H, s, CH₃), 2.36, 3.38 (6H, 2s, 2 OCH₃), 2.66-2.83 (2H, m, 2′-H), 2.96, 3.15 (6H, 2 s, 2 NCH₃), 4.48-4.60 (2H, m, 5′-H and 4′-H), 5.63 (1H, m, 3′-H), 6.14 (2H, s, OCH₂), 6.98 (1H, t, J 6.7 Hz, 1′-H), 7.30-7.37 (4H, m, arom. H), 7.81-7.92 (6H, m, arom. H and 6-H), 8.50 (1H, s, N═CH).

### 5-[2-Deoxy-β-D-erythro-pentofuranosyl]-{[2-(dimethylamino)methylidene]amino}-3,5-dihydro-7-prop-1-ynyl)-4H-pyrrolo[3,2-d]pyrimidin-4-one (19)

The solution of 18 (1 g, 1.40 mmol) in 0.1 M NaOMe in MeOH (50 ml) was stirred for 1 h at r. t. The reaction mixture was cooled and neutralized with 5% acetic acid in MeOH. Silica gel was added (4 g) and the solvent was evaporated. It was applied to FC (silica gel, column 14×3 cm, CH₂Cl₂/MeOH, 9:1). Main zone afforded 19 as a colorless solid (480 mg, 95%) (Found: C, 58.80;H, 5.74; N, 19%. C₁₇H₂₁N₅O₄ requires C, 56.82; H, 5.89; N, 19.49%); TLC (silica gel, CH₂Cl₂/MeOH, 95:5): R_{f}0.20; λₘₐₓ (MeOH)/nm 226 (ε/dm³ mol⁻¹ cm⁻¹ 25800), 266 (23300) and 300 (19200); δ_{H} (250.13 MHz; [d₆]DMSO; Me₄Si) 2.03 (3H, s, CH₃), 2.21-2.28 (2H, m, 2′-H), 3.00, 3.14 (6H, 2 s, 2 NCH₃), 3.51 (2H, m, 5′-H), 3.79 (1H, m, 4′-H), 4.28 (1H, m, 3′-H), 4.94 (1H, t, J 5.53 Hz, 5′-OH), 5.21 (1H, d, J 3.80 Hz, 3′-OH), 6.85 (1H, t, J 6.75 Hz, 1′-H), 7.80 (1H, s, 6-H), 8.50 (1H, s, N═CH), 11.25 (1H, s, NH).

### 5-[2-Deoxy-β-D-erythro-pentofuranosyl]-2-{[(dimethylamino)methylidene]amino}-3,5-dihydro-3-[(pivaloyloxy)methyl]-7-(prop-1-ynyl)-4H-pyrrolo[3,2-d]pyrimidin-4-one (20)

The solution of compound 18 (400 mg, 0.56 mmol) in 0.01 M NaOMe/MeOH (20 ml) was stirred for 45 min at r. t. The reaction mixture was cooled in ice bath and neutralized with 5% acetic acid in MeOH. Silica gel (2 g) was added and the solvent was evaporated to dryness. The resulting powder was applied to FC (silica gel, column 14×2 cm, CH₂Cl₂/MeOH, 98:2). Main zone afforded compound 20 as a colorless solid (158 mg, 59%) (Found: C, 58.26; H, 6.70; N, 14.61%. C₂₃H₃₁N₅O₆ requires C, 58.34; H, 6.60; N, 14.79%). TLC (silica gel, CH₂Cl₂/MeOH, 95:5): R_{f}0.36; λₘₐₓ (MeOH)/nm 218 (ε/dm³ mol⁻¹ cm⁻¹ 41500), 267 (38400) and 312 (29100); δ_{H} (250.13 MHz; [d₆]DMSO; Me₄Si) 1.05 (9H, s, 3 CH₃), 1.99 (3H, s, CH₃), 2.20-2.25 (2H, m, 2′-H), 2.94, 3.13 (6H, 2 s, 2 NCH₃), 3.50 (2H, m, 5′-H), 3.76 (1H, m, 4′-H), 4.25 (1H, m, 3′-H), 4.94 (1H, t, J4.94 Hz, 5′-OH), 5.21 (1H, d, J 3.65 Hz, 3′-OH), 6.12(2H, s, OCH₂), 6.80 (1H, t, J 6.45 Hz, 1′-H), 7.85 (1H, s, 6-H), 8.47 (1H, s, N═CH).

### 5-[2-Deoxy-5-O-(4,4′-dimethoxytrityl)-β-D-erythro-pentofuranosyl]-2-{[(dimethylamino)methylidene]amino}-3,5-dihydro-7-(prop-1-ynyl)-4H-pyrrolo[3,2-d]pyrimidin-4-one (21)

Compound 19 (170 mg, 0.47 mol) was dried by repeated co-evaporation with anhydrous pyridine (2×3 ml) and dissolved in pyridine (4 ml). After addition of 4,4′-dimethoxytrityl chloride (170 mg, 0.50 mmol) a solution was stirred for 2 h at r. t. CH₂Cl₂ (30 ml) was added and washed with 5% eq. NaHCO₃ soln. (25 ml). The aq. layer was extracted with CH₂Cl₂ (25×2 ml). The combined organic phase was dried over Na₂SO₄ and evaporated to dryness. The residue was subjected to FC (silica gel, column 12×2 cm, CH₂Cl₂/(CH₃)₂CO, 95:5). Main zone afforded compound 21 as a colorless foam (225 mg, 72%) (Found: C, 68.79;H, 5.91; N, 10.39%. C₃₈H₃₉N₅O₆ requires C, 68.97; H, 5.94; N, 10.58); TLC (silica gel, CH₂Cl₂/MeOH, 95:5): R_{f}0.27; λₘₐₓ (MeOH)/nm 231 (ε/dm³ mol⁻¹ cm⁻¹ 40600), 266 (25300) and 301 (18300); δ_{H} (250.13 MHz; [d₆]DMSO; Me₄Si) 1.99 (3H, s, CH₃), 2.24 (1H, m, 2′-H_{α}), 2.34 (1H, m, 2′-H_{β}), 2.99 (3 H, s, NCH₃), 3.12 (5H, m, NCH₃ and 5′-H), 3.72 (6H, s, 2 OCH₃), 3.88 (1H, m, 4′-H), 4.27 (1H, m, 3′-H), 5.31 (1H, d, J 4.07 Hz, 3′-OH), 6.84 (5H, m, 1′-H and arom. H), 7.23-7.38 (9H, m, arom. H), 7.61 (1H, s, 6-H), 8.49 (1H, s, N═CH), 11.28 (1H, s, NH).

### 5-[2-Deoxy-5-O-(4,4′-dimethoxytrityl)-β-D-erythro-pentofuranosyl]-2-{[(dimethylamino)methylidene]amino}-3,5-dihydro-3-[(pivaloyloxy)methyl]-7-(prop-1-ynyl)-4H-pyrrolo[3,2-d]pyrimidin-4-one (22)

Compound 20 (170 mg, 0.36 mmol) was dried by repeated co-evaporation with anhydrous pyridine (2×3 ml) and dissolved in pyridine (4 ml). After addition of 4,4′-dimethoxytrityl chloride (135 mg, 0.39 mmol) a soln. was stirred for 2 h at r. t. and CH₂Cl₂ (30 ml) was added and washed with 5% aq. NaHCO₃ soln. (25 ml). The aq. layer was extracted with CH₂Cl₂ (25×2 ml), dried over Na₂SO₄ and evaporated to dryness. The residue was subjected to FC (silica gel, column 12×2 cm, CH₂Cl₂/(CH₃)₂CO, 95:5). Main zone afforded compound 22 as a colorless foam (210 mg, 76%) (Found: C, 68.16; H, 6.37; N, 9.01%. C₄₄H₄₉N₅O₈ requires C, 68.11; H, 6.37; N, 9.03%); TLC (silica gel, CH₂Cl₂/MeOH, 95:5): R_{f}0.42; λₘₐₓ (MeOH)/nm 231 (ε/dm³ mol⁻¹ cm⁻¹ 41100), 269 (28700) and 313 (19800); δ_{H}(250.13 MHz; [d₆]DMSO; Me₄Si) 1.09 (9H, s, 3 CH₃), 2.01 (3H, s, CH₃), 2.24 (1H, m, 2′-H_{α}), 2.38 (1H, m, 2′-H_{β}), 2.98 (3H, s, NCH₃), 3.16 (5H, m, NCH₃ and 5′-H), 3.73 (6H, s, 2 OCH₃), 3.91 (1H, m, 4′-H), 4.29 (1H, m, 3′-H), 5.29 (1H, d, J 4.25 Hz, 3′-OH), 6.17 (2 s, OCH₂), 6.84 (5H, m, 1′-H and arom. H), 7.20-7.39 (9H, m, arom. H), 7.72 (1H, s, 6-H), 8.52 (1H, s, N═CH).

### 5-[2-Deoxy-5-O-(4,4′-dimethoxytrityl)-β-D-erythro-pentofuranosyl]-2-{[(dimethylamino)methylidene]amino}-3,5-dihydro-7-(prop-1-ynyl)-4H-pyrrolo[3,2-d]pyrimidin-4-one 3′-[2-Cyanoethyl-diisopropylphosphoramidite] (23)

To a soln. of compound 21 (145 mg, 0.22 mmol) in anhydrous CH₂Cl₂ (5 ml), N,N-diisopropylethylamine (DIPEA) (70 μl, 0.40 mmol) and 2-cyanoethyl-diisopropylphosphoramido chloridite (84 μl, 0.37 mmol) were added under Ar atmosphere. After stirring for 30 min, 5% aq. NaHCO₃ soln. was added, and it was extracted with CH₂Cl₂ (10 ml×2). The org. layer was dried over Na₂SO₄, filtered and evaporated. The residue was subjected to FC (silica gel, CH₂Cl₂/acetone, 9:1). Main zone afforded compound 23 as a colorless foam (140 mg, 74%). TLC (silica gel, CH₂Cl₂/(CH₃)₂CO, 8:2): R_{f}0.6; ³¹P-NMR (CDCl₃): δ 149.79, 150.12.

### 5-[2-Deoxy-5-O-(4,4′-dimethoxytrityl)-β-D-erythro-pentofuranosyl]-2-{[(dimethylamino)methylidene]amino}-3,5-dihydro-3-[(pivaloyloxy)methyl]-7-(prop-1-ynyl)-4H-pyrrolo[3,2-d]pyrimidin-4-one 3′-[2-Cyanoethyl-diisopropylphosphoramidite] (24)

As described for 21 with compound 22 (215 mg, 0.28 mmol), N,N-diisopropylethylamine (DIPEA) (40 μl, 0.23 mmol) and 2-cyanoethyl-diisopropylphosphoramido chloridite (50 μl, 0.22 mmol) in anhydrous CH₂Cl₂ (5 ml). FC (silica gel, CH₂Cl₂/acetone, 9:1) afforded compound 24 as a colorless foam (120 mg, 44%). TLC (silica gel, CH₂Cl₂/(CH₃)₂CO, 8:2): R_{f}0.7; ³¹P-NMR (CDCl₃): δ 149.79, 150.12.

### Example 14

### Synthesis of propynyl-2′-deoxycytidine

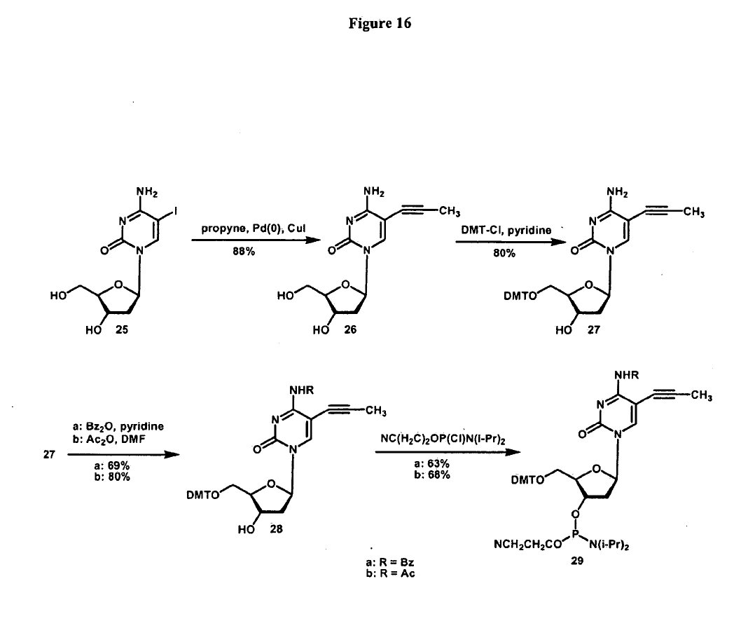

The synthesis of 2′-deoxycytidine analogues resembling the donor/acceptor pattern of 2′deoxycytidine was performed according to FIG. 16.

### 5-Propynyl-2′-deoxycytidine (26)

5-Iodo-2′-deoxycytidine (6.0 g, 17 mmol) (25) was dissolved in dry DMF (80 ml) under argon. Copper(I)iodide (99.99%, 652 mg, 3.3 mmol), tetrakis(triphenylphosphin)palladium(0) (1.7 g, 1.5 mmol) and triethylamine (4.8 ml, 3.4 g, 34.5 mmol) were added. The mixture was cooled in an ice-bath and propyne gas (99%) was added with slightly bubbling during a period of 20 min under cooling. The reaction mixture was stirred overnight at r.t. The TLC (CH₃CN—H₂O 95:5 containing 1 Pipette of 25% aq. NH₃ on 100 ml of solvent) showed a homogenous zone (2 developments) moving a little bit faster then the starting material. The solvent was evaporated)(70°) and coevaporated with toluene (3×50 ml). The solid residue was suspended in CH₂Cl₂ and precipated thereof. The solid material was filtrated and washed carefully with CH₂Cl₂ furnishing compound 26 (3.9 g, 88%) as crude product. It was directly used in the next step. TLC (CH₂Cl₂:MeOH 19:1, 2 developments) R_{f}0.30. ¹H-NMR ((D₆)DMSO): δ =2.0 (m, CH₃ and H—C(2′)); 3.57 (m, H—C(5′)); 3.78 (m, H—C(4′)); 4.19 (m, H—C(3′)); 5.07 (m, HO—C(5′)); 5.20 (m, HO—C(3′)); 6.11 (‘t’, J=6.0, H—C(1′)); 6.94 and 7.72 (2s, 2NH); 8.08 (s, H—C(6)).

### 5′-O-(4,4′-Dimethoxytriphenylmethyl)-5-(1-propynyl)-2′-deoxycytidine (27)

Compound 26 (5.0 g, 18.8 mmol) was dissolved in dry pyridine (20 ml) and treated with 4,4′-dimethoxytriphenylmethylchloride (6.0 g, 17.7 mmol). The reaction mixture was stirred overnight, diluted with CH₂Cl₂ (30 ml) and the reaction quenched with 5% NaHCO₃-solution (40 ml). The aq. layer was extracted with CH₂Cl₂ (3×40 ml) and the combined org. layers were dried (Na₂SO₄), filtrated and the solvent evaporated. The oily residue was coevaporated with toluene (3×50 ml) and then applied to FC (silica gel, column 15×4 cm, CH₂Cl₂→CH₂Cl₂:MeOH 98:2→CH₂Cl₂:MeOH 95:5→CH₂Cl₂:MeOH 9:1) furnishing compound 27 (8.5 g, 80%) as colorless solid. TLC (CH₂Cl₂:MeOH 9:1) R_{f}0.5. ¹H-NMR ((D₆)DMSO): δ=1.84 (m, CH₃); 2.11 (m, H—C(2′)); 3.11 and 3.23 (2m, 2H—C(5′)); 3.74 (OCH₃); 3.93 (m, H—C(4′)); 4.26 (m, H—C(3′)); 5.32 (m, HO—C(3′)); 6.14 (n, H—C(1′)); 6.90 (m, arom.H and NH of NH₂); 7.32 (m, arom.H); 7.72 (s, NH of NH₂); 7.88 (s, H—C(⁶)).

### 4-N-Benzoyl-5′-O-(4,4′-dimethoxytrityl)-5-(1-propynyl)-2′-deoxycytidine (28a)

Compound 27 (1.7 g, 3.0 mmol) was dissolved in anh. pyridine (20 ml). Then, N-ethyldiisopropylamine (1.8 ml, 10.4 mmol) and benzoic anhydride (1.15 g, 5.1 mmol) were added and the solution was stirred overnight at r.t. The solvent was evaporated and the oily residue was coevaporated with toluene (3×20 ml). The resulting foam was applied to FC (silica gel, column 15×4 cm, CH₂Cl₂→CH₂Cl₂:acetone9:1→CH₂Cl₂:acetone 4:1→CH₂Cl₂:acetone 1:1) furnishing compound 28a (1.4 g, 69%) as colorless solid. TLC (CH₂Cl₂:acetone 1:1) R_{f}0.7. ¹H-NMR ((D₆)DMSO): δ =1.69 (s, CH₃); 2.29 (m, H—C(2′)); 3.24 (m, 2H—C(5′)); 3.74 (OCH₃); 4.00 (m, H—C(4′)); 4.29 (m, H—C(3′)); 5.38 (m, HO—C(3′)); 6.12 (n, H—C(1′)); 6.90 (m, arom.H and NH of NH₂); 7.32 (m, arom.H); 8.01 (m, NH of NH₂.H—C(6)); 12.56 (bs, NH).

### 4-N-Benzoyl-5′-O-(4,4′-dimethoxytrityl)-5-(1-propynyl)-2′-deoxycytidine 3′-[(2-cyanocthyl)-N,N-(diisopropyl)]phosphoramidite (29a)

Compound 28a (750 mg, 1.1 mmol) was dissolved in dry CH₂Cl₂ (10 ml). The mixture was treated with N-ethyldiisopropylamine (0.4 ml, 2.3 mmol) and 2-cyanoethyl diisopropylphosphoramidochloridite (0.4 ml, 1.8 mmol) for 20 min at r.t. “The solution was diluted with CH₂Cl₂ (10 ml) and poured into 50 ml 5% NaHCO₃-soln. The aq. layer was extracted with CH₂Cl₂ (3×50 ml), the combined org. layers were dried (Na₂SO₄), filtrated and evaporated. The residual foam was purified by FC (silica gel, column 10×5 cm, CH₂Cl₂/acetone 9:1). Evaporation of the main zone afforded compound 29a (600 mg, 63%) as yellowish foam. TLC (CH₂Cl₂:acetone 9:1) R_{f}0.7. ¹H-NMR ((D₆)DMSO): ³¹P-NMR (CDCl₃): 149.8, 150.3. Anal. calc. for C₄₉H₅₄N₅O₈P (871.96) calc.: C 67.49 H 6.24 N 8.03; found: C 67.65 H 6.29 N 7.80.

### 4-N-Acetyl-5′-O-(4,4′-dimethoxytrityl)-5-(1-propynyl)-2′-deoxycytidine (28b)

Compound 27 (5.0 g, 8.8 mmol) was dissolved in dry N,N-dimethylformamide (50 ml). Then, acetic acid anhydride (1.0 ml, 10.6 mmol) was added and the solution was stirred overnight at r.t. The solvent was evaporated and the oily residue was co-evaporated with toluene (3×20 ml). The resulting foam was applied to FC (silica gel, column 15×4 cm, CH₂Cl₂→CH₂Cl₂:acetone9:1→CH₂Cl₂:acetone 4:1→CH₂Cl₂:acetone 1:1) furnishing compound 28b (4.3 g, 80%) as colorless foam. TLC (CH₂Cl₂:acetone 1:1) R_{f}0.5. ¹H-NMR ((D₆)DMSO): δ=1.84 (s, CH₃); 2.20 (m, H—C(2′)); 2.35 (s, CH₃) 3.24 (m, 2H—C(5′)); 3.74 (OCH₃); 4.02 (m, H—C(4′)); 4.28 (m, H—C(3′)); 5.37 (d, J=4.25, HO—C(3′)); 6.06 (‘t’, J=6.2, H—C(1′)); 6.90 and 7.3 (2m, arom.H and NH₂); 7.32 (m, arom.H); 8.20 (s, H—C(6)); 9.2 (s, NH).

### 4-N-Acetyl-5′-O-(4,4′-dimethoxytrityl)-5-(1-propynyl)-2′-deoxycytidine 3′-[(2-cyanoethyl)-N,N-(diisopropyl)]phosphoramidite (29b)

Compound 28b (4.1 g, 6.7 mmol) was dissolved in anh. CH₂Cl₂ (10 ml). The mixture was treated with N-ethyldiisopropylamine (3.1 ml, 17.8 mmol) and 2-cyanoethyl diisopropylphosphoramidochloridite (3.1 ml, 14.0 mmol) for 20 min at r.t. The solution was diluted with CH₂Cl₂ (10 ml) and poured into 50 ml 5% NaHCO₃-soln. The aq. layer was extracted with CH₂Cl₂ (3×50 ml), the combined org. layers were dried (Na₂SO₄), filtrated and evaporated. The residual foam was purified by FC (silica gel, column 10×5 cm, CH₂Cl₂/acetone 4:1). Evaporation of the main zone afforded compound 29b (3.7 g, 68%) as white foam. TLC (CH₂Cl₂:acetone 4:1) R_{f}0.7. ¹H-NMR ((D₆)DMSO): ³¹P-NMR (CDCl₃): 149.8, 150.4. Anal. calc. for C₄₄H₅₂N₅O₈P (809.89) calc.: C 65.25 H 6.47 N 8.65; found: C 65.18 H 6.39 N 8.70.

What is claimed is:

1. A bioconjugate comprising at least one nanoparticle bound to at least one nucleic acid binding compound having a backbone, wherein the at least one nucleic acid binding compound is adapted to form an i-motif structure or an i-motif related structure, thus forming a composition.

2. The bioconjugate of claim 1, wherein a plurality of nucleic acid binding compounds which are capable of forming i-motif structures or i-motif related structures are bound to the nanoparticle.

3. The bioconjugatc according to claim 1, wherein the i-motif structure or the i-motif related structure has the formula 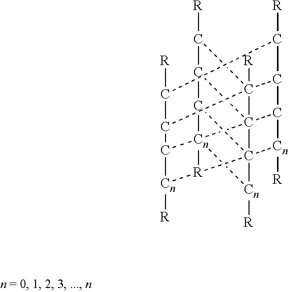
wherein R=a residue and C=carbon atom.

4. The bioconjugate according to claim 1, wherein a chain of the at least one nucleic acid binding compound in the i-motif structure or in the i-motif related structure is held to another chain by base-pairs selected from the group consisting of the following motifs 1 to 3 wherein B=backbone:

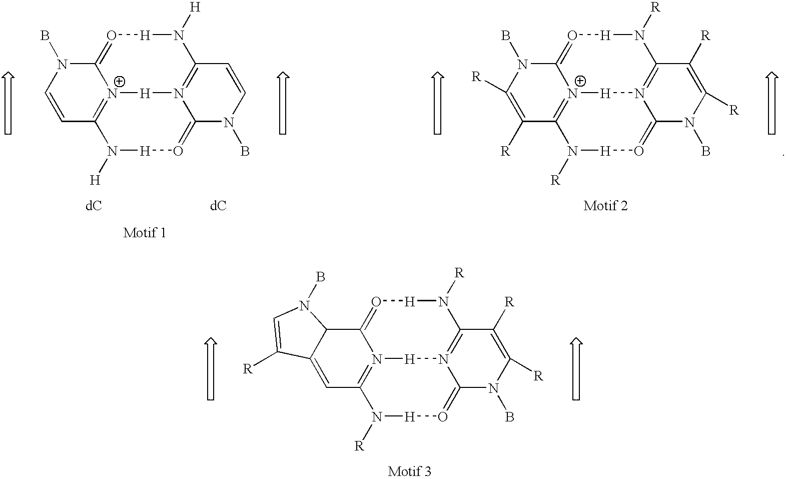

5. The bioconjugate according to claim 1, further comprising at least a further nucleic acid binding compound associated with the at least one nucleic acid binding compound to form the composition.

6. The bioconjugate according to claim 1 wherein the backbone has attached heterocycles capable of forming an i-motif structure or an i-motif related structure.

7. The bioconjugate according to claim 6, wherein the attached heterocycles are selected from the group consisting of the following formulae 1-5: 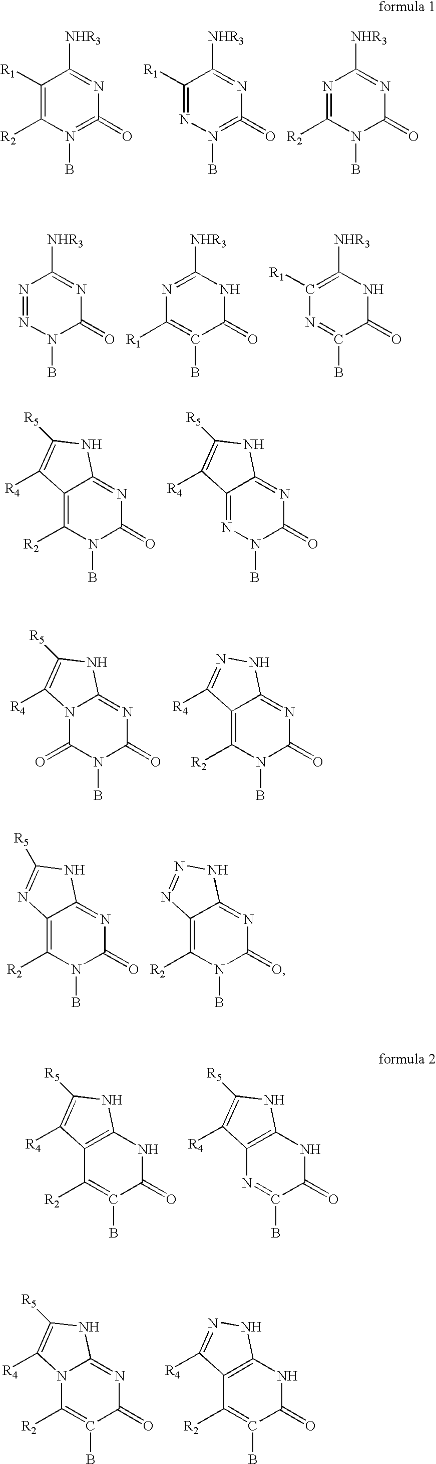 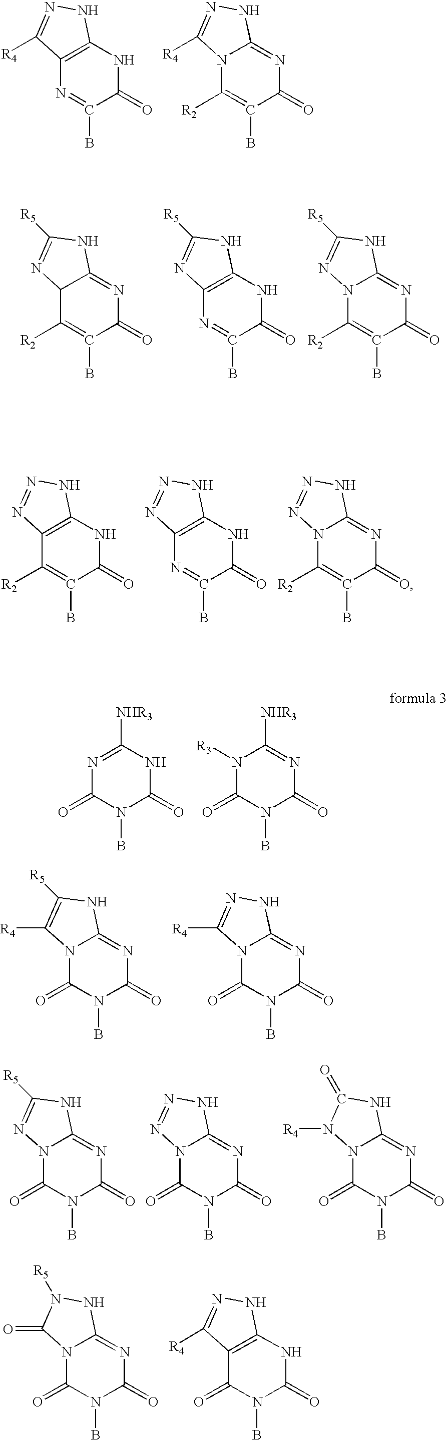 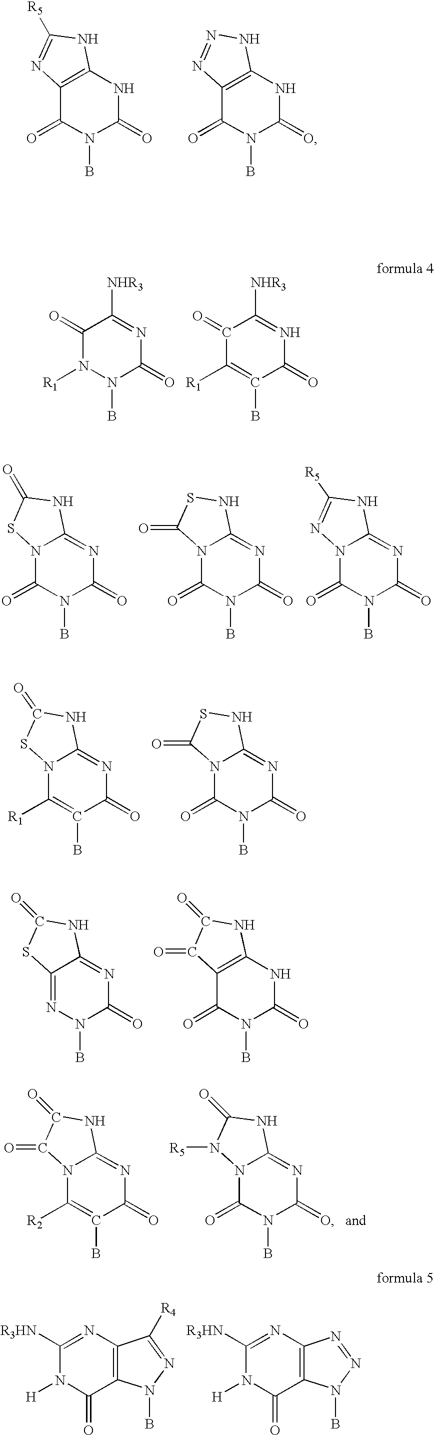 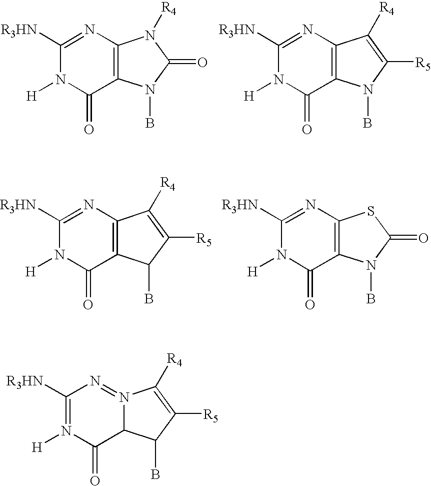
wherein
R₁, R₂, R₄, and R₅ are independent from each other and are independent from R₃;
R₁, R₂, R₄, and R₅ are selected from the group consisting of
—H,
—F, —Cl, —Br, or —I,
nitro,
amino,
cyano,
—COO—,
(C₁-C₅₀)-alkyl substituted according to (12),
(C₂-C₅₀)-alkenyl substituted according to (12),
(C₂-C₅₀)-alkynyl substituted according to (12),
(C₆-C₅₀)-aryl substituted according to (12),
—W—(C₁-C₅₀)-alkyl, —W—(C₁-C₅₀)-alkenyl, —W—(C₁-C₅₀)-alkynyl, —W—(C₆-C₅₀)-aryl or W—H, wherein W=—S—, —O—, —NH—, —S—S—, —CO—, —COO—, —CO—NH—, —NH—CO—, —NH—CO—NH—, NH—CS—NH—, —(CH₂)ₙ-[O—(CH₂)ₜ]ₛ—, where r and s are, independently of each other, an integer between 1 to 18 and n is 0 or 1 independently from r and s,
substituents (7) to (11) wherein any alkyl, alkenyl, alkynyl or aryl can be substituted by one or more moieties selected from the group consisting of -halogen, —SH, —NO₂, —CN, —S—(C₁-C₆)-alkyl, —(C₁-C₆)-alkoxy, —OH, NR⁶R⁷, —N′R⁶R⁷R⁸, —OR¹², —COR⁹, —NH—CO—NR⁶R⁷, —NH—CS—NR⁶R⁷, and —(CH₂)ₙ-[O—(CH₂)ᵣ]ₛ—NR⁶R⁷ where r and s are, independently of each other, an integer between 1 to 18 and n is 0 or 1 independently from r and s, wherein R⁹ is selected from the group consisting of —OH, —(C₁-C₆)-alkoxy, —(C₆-C₂₂)-aryloxy, —NHR⁸, —OR⁸, —SR⁸, wherein R⁶, R⁷, and R⁸ are selected independently from the group consisting of —H, —(C₁-C₁₀)-alkyl, —(C₁-C₁₀)-alkenyl, —(C₁-C₁₀)-alkynyl, —(C₆-C₂₂)-aryl and a reporter group or a group which facilitates intracellular uptake said alkyl, alkenyl, alkynyl or aryl in substituents (7) to (12) being unsubstituted or substituted by one or more moieties selected from the group consisting of -halogen, —SH, —S—(C₁-C₆)-alkyl, —(C₁-C₆)-alkoxy, —OH, NR⁶R⁷, —COR⁹, —NH—CONR⁶R⁷, —NH—CSNR⁶R⁷, and —(CH₂)ₙ-[O—(CH₂)ᵣ]ₛ—NR⁶R⁷ where r and s are, independently of each other, an integer between 1 to 18 and n is 0 or 1 independently from r and s;
R₃ is independent from R₁, R₂, R₄, or R₅ and is selected from the group consisting of:
—H,
(C1-C50)-alkyl,
(C2-C50)-alkenyl,
(C2-C50)-alkynyl,
(C6-C50)-aryl,
(C6-C50)-aryloxy,
—Z—(C1-C50)-alkyl, —Z—(C1-C50)-alkenyl, —Z—(C1-C50)-alkynyl, —Z—(C6-C50)-aryl or Z—H, wherein Z=—CO—, —CO—NH—, —CS—NH—, —(CH2)n-[O—(C1-C12)r]s-, where r and s are, independently of each other, an integer between 1 to 18 and n is 1 or 2 independently from r and s,
substituents (2) to (7) wherein any alkyl, alkenyl, alkynyl or aryl can be substituted by one or more moieties selected from, the group consisting of -halogen, NO2, —OR8, —CN, —SH, —S—(C1-C6)-alkyl, —(C1-C6)-alkoxy, —OH, NR6R7, —N+R6R7R8, —COR9, —NH—CONR6R7, —NH—CSNR6R7, and —(CH2)n-[O—(CH2)r]s-NR6R7 where r and s are, independently of each other, an integer between 1 to 18 and n is 0 or 1 independently from r and s, wherein R9 is selected from the group consisting of —OH, —(C1-C6)-alkoxy, —(C6-C22)-aryloxy, —NHR8, —OR8, —SR8, wherein R6, R7, and R8 are selected independently from the group consisting of —H, —(C1-C10)-alkyl, -(C1-C10)-alkenyl, —(C1-C10)-alkynyl, —(C6-C22)-aryl and a reporter group, said alkyl, alkenyl, alkynyl or aryl in substituents (2) to (8) being unsubstituted or substituted by one or more moieties selected from the group consisting of -halogen, —SH, —S—(C1-C6)-alkyl, —(C1-C6)-alkoxy, —OH, NR6R7, —COR9, —NH—CONR6R7, —NH—CSNR6R7, and —(CH2)n-[O—(CH2)r]s-NR6R7 where r and s are, independently of each other, an integer between 1 to 18 and n is 0 or 1 independently from r and s; and
B is the position of attachment of the group to the backbone of the nucleic acid binding compound; and any salts thereof.

8. The bioconjugate according to claim 6 wherein the attached heterocycle displays a donor/acceptor pattern characteristic of natural cytosine.

9. The bioconjugate according to claim 1 further comprising a non-heterocyclic residue which displays a donor/acceptor pattern characteristic for natural cytosine.

10. The bioconjugate according to claim 6 further comprising tautomeric forms and salts of the attached heterocycles.

11. The bioconjugate according to claim 1, wherein the at least one nucleic acid binding compound comprises one or more moieties having the formula: 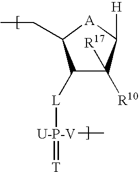
wherein
A is selected from the group consisting of O, S, Se, Te, CH2, and N—CO—(C1-C50)-alkyl,
L is selected from the group consisting of oxy, sulfanediyl, —CH2—, and —NR11—,
T is selected from the group consisting of oxo, thioxo, selenoxo, and telluroxo,
U is selected from the group consisting of —OH, O—, —O-reporter group, —SH, —S, reporter group, —SeH, —(C1-C50)-alkoxy, —(C1-C50)-alkyl, —(C6-C50)-aryl, —(C6-C50)-aryl-(C1-C50)-alkyl, —NR12R13, and —(—O—(C1-C50)-alkyl-)n-R14, wherein n can be any integer between 1 and 6, or wherein —NR12R13 can together with N be a 5-6-membered heterocyclic ring,
V is selected from the group consisting of oxy, sulfanediyl, —CH2—, and —NR11—,
R10 and R17 are independently selected from the group consisting of —H, —OH, —(C1-C50)-alkyl, —(C1-C50)-alkenyl, —(C1-C50)-alkynyl, —(C1-C50)-alkoxy, —(C2 -C50)-alkenyloxy, —(C2-C50)-alkynyloxy, -halogen, -azido, —O-alkyl, —O-allyl, and —NH2,
R11 is independently selected from the group of —H and —(C1-C10)-alkyl,
R12 and R13 are independently selected from the group consisting of —(C1-C50)-alkyl, —(C1-C50)-aryl, —(C6-C50)-aryl-(C1-C50)-alkyl, —(C1-C50)-alkyl-[NH(CH2)c]d-NR15R16 and a reporter group,
R14 is selected from the group consisting of —H, —OH, -halogen, -amino, —(C1-C50)-alkylamino, —COOH, —CONH2 and —COO(C1-C50)-alkyl and a reporter group,
R15 and R16 are independently selected from the group consisting from —H, —(C₁-C₅₀)-alkyl, and —(C₁-C₅₀)-alkoxy-(C₁-C₅₀)-alkyl and a reporter group, and
H is a heterocycle showing the donor/acceptor pattern of cytosine.

12. The bioconjugate of claim 1, wherein the backbone comprises one or more sugar moieties and one or more phosphate moieties.

13. The bioconjugate according to claim 12 wherein the one or more sugar moieties exhibit an α-D-, β-D-, α-L- and/or β-L-configuration or a parallel or anti-parallel chain orientation.

14. The bioconjugate according to claim 12 wherein the one or more sugar moieties are connected to the attached heterocycle via a N-glycosylic or C-glycosylic bond.

15. The bioconjugate according to claim 12 wherein the one or more sugar moieties are in a locked conformation.

16. The bioconjugate according to claim 1, wherein the at least one nucleic acid binding compound contains a reporter group.

17. The bioconjugate according to claim 1 comprising a structure having the formula 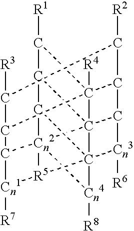
wherein
represents a connector of any backbone within the i-motif structure or i-motif related structure;
C represents cytosine residues or derivatives thereof displaying the donor/acceptor pattern of cytosine;
R₁-R₈ are independently from each other with the proviso that at least one of these residues R₁-R₈ is the at least one nanoparticle and the remaining residues are selected from the group consisting of:
any naturally occurring or artificial backbone connected to the i-motif,
oligonucleotides including modified oligonucleotides,
DNA, RNA, LNA, PNA in which one or more sugar moieties exhibit the α-D-, β-D-, α-L- and/or β-L-configuration,
nanoparticle,
micro-particle and/or any larger particle,
protecting group,
surface;
reporter group,
linker and connector unit,
dendrimeric structure,
stiff linkers, e.g., formed by incorporation of triple bonds,
multi-linker units,
spacer unit,
linker unit connecting at least two strands of the i-motif with each other forming hairpin structures,
attachment unit,
antibody,
antigenic group,
linker, spacer and/or reporter units with the capability to generate non-covalent interactions (e.g., the biotin-avidin system, antigen-antibody interaction),
delivery unit (e.g., steroids, liposomes),
linker, spacer and/or reporter unit with the capability to form covalent interactions via the Huisgen-Sharpless cycloaddition “click-chemistry”, and
—H; and
n¹-n⁴ are independ from each other and are integers between 0 and n.

18. The bioconjugate according to claim 17, wherein the residues of R₁-R₈ can be connected in any order or in any combination.

19. The bioconjugate according to claim 17, wherein R₁-R₈ form higher ordered structures such as hairpins, triplexes, or/and quadruplexes.

20. The bioconjugate according to claim 1 further comprising a further nanoparticle.

21. The bioconjugate according to claim 1 further comprising a protecting group.

22. The bioconjugate according to claim 1, wherein the at least one nucleic acid binding compound is attached to a surface.

23. The bioconjugate according to claim 1, wherein the at least one nucleic binding compound has an artificial backbone.

24. A bioconjugate according to claim 1, wherein the at least one nanoparticle is bound to the at least one nucleic binding compound via a linker or connector unit.

25. The bioconjugate according to claim 1, wherein the bioconjugate is attached to a dendrimeric structure.

26. The bioconjugate according to claim 17, wherein the linker is a stiff linker.

27. The bioconjugate according to claim 17, wherein the linker is a multi-linker unit.

28. The bioconjugate according to claim 17, wherein the linker is attached to a spacer unit.

29. The bioconjugate according to claim 17, wherein the linker connects at least two strands of the i-motif structure or the i-motif related structure, thus forming hairpin-like structures.

30. The bioconjugate according to claim 17 further comprising an antibody.

31. The bioconjugate according to claim 17 further comprising an antigenic group.

32. The bioconjugatc according to claim 1, wherein the formed composition is attached to linker, spacer and/or reporter unit with the capability to generate non-covalent interactions.

33. The bioconjugate according to claim 1 further comprising a delivery unit.

34. The bioconjugate according to any claim 1, wherein the bioconjugate is attached to linker, spacer and/or reporter unit with the capability to form covalent interactions via the Huisgen-Sharpless cycloaddition “click-chemistry”.

35. The bioconjugate according to claim 1, further comprising a stabilizer used to increase the stability of said formed composition.

36. The bioconjugate according to claim 6, further comprising a modified one of the attached heterocycles used to increase the stability of the formed composition.

37. The bioconjugate according to claim 1, further comprising a modified one of the backbone used to increase the stability of the formed composition.

38. A method for the detection of an amount of a compound in a sample comprising the steps of
providing a sample suspected to contain the compound,
contacting a bioconjugate with the sample under conditions allowing the formation of i-motif structure or an i-motif related structure between the bioconjugate and the compound, wherein the bioconjugate comprises at least one nanoparticle bound to at least one nucleic acid binding compound having a backbone,
determining a degree of assembling of the bioconjugate and the compound, whereby the degree of assembling is indicative of the amount of the compound in the sample.

39. The method according to claim 38, further comprising the step of changing the pH of the sample, thereby changing the degree of assembling between the bioconjugate and the compound.

40. The method according to claim 38, further comprising the step of changing the temperature of the sample, thereby changing the degree of assembling between the bioconjugate and the compound.

41. A kit for the detection of an amount of a compound in a sample according to the method of claim 38, the kit comprising:
a container holding at least a bioconjugate comprising at least one nanoparticle bound to at least one nucleic acid binding compound having a backbone wherein the at least one nucleic acid binding compound is adapted to form an i-motif structure or an i-motif related structure.

42. The kit according to claim 41, wherein a disassembly of the i-motif structure or i-motif related structure formed between the bioconjugate and the compound to be detected can be achieved by pH-changes.

43. The kit according to claim 41, wherein the disassembly of the i-motif structure or i-motif related structure formed between the bioconjugate and the compound to be detected can be achieved by temperature changes.

44. A method in which a bioconjugate according to claim 1 acts as a nanomachine, the method comprising the steps of
providing at least the bioconjugate, and
changing the pH of the bioconjugate such as to form reversibly the i-motif structure or the i-motif related structure.

45. The method according to claim 44 wherein the bioconjugate is capable of forming i-motif structures or i-motif related structures between at least the bioconjugate and at least a further nucleic acid binding compound.

46. A method in which a composition formed from a bioconjugate according to claim 1 is used as a pH-sensitive colorimetric sensor, the method comprising the steps of
providing at least the bioconjugate, wherein the at least one nanoparticle is a gold nanoparticle,
detecting colorimetric changes caused by the formation or disassembly of the composition.

47. A method in which a bioconjugate according to claim 1 is used for the detection of tumour cells at a site suspected to be diseased, the method comprising the steps of
providing at least the bioconjugate to the site suspected to be diseased,
observing the presence and/or absence of said i-motif structures or i-motif related structures formed by the bioconjugate at the site suspected to be diseased.

48. The method according to claim 47 further comprising the step of determining a degree of assembly of the i-motif structures or i-motif related structures formed by the bioconjugate at the diseased site.

49. The method according to claim 48, further providing a method for the detection of the reporter group.

50. A method for the treatment of tumour tissue at diseased sites using a bioconjugate according to claim 1, the method comprising the steps of
providing the bioconjugate to the tumour tissue,
observing the formation of the i-motif structures or i-motif related structures at the diseased sites,
and at least partially destroying the tumour tissue marked by the formation of the i-motif structures or the i-motif related structures.

51. The method according to claim 50 comprising wherein the at least one nanoparticle is capable of being irradiated.

52. The method according to claim 50, further including a step of the irradiation of the sites.

53. The method according to claim 50, wherein x-rays irradiate the sites.

54. The method according to claim 50, wherein a magnetic field or an electric field irradiate the sites.

55. The method according to claim 47 further including a step on the basis of hyperthermy for a selective heating of the sites marked by the formation of the i-motif structures or the i-motif related structures.

56. A method for the detection and treatment of a viral disease comprising the steps of
providing at least a bioconjugate according to claim 1 at the site suspected to be diseased,
observing the presence and/or absence of the i-motif structures or i-motif related structures formed by the bioconjugate at site suspected to be diseased, and
at least partially destroying the viral disease.

57. A vaccine comprising a bioconjugate according to claim 1.

58. A method for the release of drugs at a diseased site comprising the steps of
providing at least a bioconjugate according to claim 1,
providing a drug that conjugates to the bioconjugate via an acidic labile linker group forms a drug conjugate, and
injecting the drug conjugate into the diseased region, whereby the drug is released at the diseased site.

59. The method according to claim 58, wherein the diseased site is a tumour tissue.

60. The method according to claim 58, wherein the drug is an oligonucleotide.

61. A method for capturing dC-rich oligonucleotides from an oligonucleotide library comprising the steps of
providing at least a bioconjugate according to claim 1 to a solution of the oligonucleotide library, and
observing the presence or absence of said i-motif structures or i-motif related structures formed by the bioconjugate in the solution of the oligonucleotide library.

62. A method for deposition of metal nanoparticles onto a surface of a substrate comprising the steps of
treating the surface of the substrate such that regions of the surface are acidic,
providing to the surface of the substrate a solution with a bioconjugate according to claim 1, wherein the at least one nanoparticle is a metal nanoparticle,
allowing the assembly of the bioconjugatc in the i-motif or the i-motif related structure at positions where the surface is acidic, thus forming the composition, and
washing the surface of the substrate to remove excess of the solution such that assembled nanoparticles remain attached to the regions of the surface.

63. The method according to claim 62, wherein the at least one nanoparticle is a gold nanoparticle.

64. A method for the deposition of a conducting strip onto a surface of an insulating substrate comprising the steps of
providing to a region on the surface of the insulating substrate a solution with a according to claim 1, wherein the at least one nanoparticle is a metal nanoparticle, thus forming the conducting strip.

65. The method according to claim 64 wherein the region on the surface of the insulating substrate is a pattern.

66. The method according to claim 65 wherein the region forms wires on the surface of the substrate.

67. The method according to claim 66, wherein the wires form electronic circuits.

68. The method according to claim 60, further comprising the step of removing organic material while maintaining the nanoparticles on the surface.

69. A method for the deposition of a metal onto a surface in which a bioconjugate according to claim 1 is used for the controlled deposition of metal nanoparticles with antimicrobial properties such as silver on surfaces of artificial joints, bone replacements, orthopaedic replacements, surgery instrumentation or other implant coatings, the method comprising the steps of
providing the bioconjugate, wherein the at least one nanoparticle is inert towards antimicrobial degradation, and
forming a coating on the surface by the bioconjugate.

70. The method according to claim 66, wherein the nanoparticle is releasable by enzymatic cleavage.

71. A method for detecting a nanoparticle present in a bioconjugate on a surface of a substrate, wherein the bioconjugate is a bioconjugate according to claim 1 conjugated to at least one nanoparticle, the method comprising the steps of
providing to a surface of the substrate a solution of the bioconjugate, and
detecting the nanoparticle by microscopy.

72. The method according to claim 71 wherein the microscopy is selected from the group consisting of atomic force microscopy, scanning electron microscopy, and tunnel electron microscopy.

73. A method for the catalysis of a fluid phase comprising the steps of
providing the fluid phase,
providing a bioconjugate according to claim 1, wherein the at least one nanoparticle is at least one catalytic active nanoparticle,
providing a surface of a substrate,
defining regions of the surface of the substrate which are made acidic,
depositing the composition onto the surface of a substrate, and
bringing the composition into contact with the fluid phase.

74. The method according to claim 73, wherein the fluid phase is a gas phase.

75. The method according to claim 73, wherein the nanoparticle removes any pollutant from the fluid phase.

76. The method according to claim 73, wherein the nanoparticle removes any pollutant from the gas phase.

77. A method in which a bioconjugate according to claim 1 is conjugated to at least one enzyme, the method comprising the steps of
providing a fluid phase that requires catalysis to perform a chemical reaction,
providing the bioconjugate that is conjugated to the at least one enzyme,
bringing the bioconjugate into contact with the fluid phase that requires catalysis,
formation by the bioconjugate of an i-motif structure or i-motif related structure in the fluid phase, and
activation of the enzyme as a response to the formation of the i-motif structure or i-motif related structure.

78. The method according to claim 77, wherein the enzyme activation is reversible.

79. The method according claim 77, wherein the active enzyme is deactivated as a response to the formation of the i-motif structure or i-motif related structure.

80. A method for determining mismatch discrimination in a sample of nucleic acid comprising the steps of
providing at least a bioconjugate according to claim 1 to the sample nucleic acid, and
observing the presence and/or absence of said i-motif structures or i-motif related structures formed by the bioconjugate in the solution of the sample of nucleic acid.

81. A method for increasing sensitivity and fidelity of nucleic acid amplification or for detection of a nucleic acid by a PCR reaction using a bioconjugate according to claim 1, the method comprising the steps of
combining the bioconjugate with the nucleic acid, and
observing the presence or absence of said i-motif structures or i-motif related structures formed by the bioconjugate to the nucleic acid.

82. A method for forming a micro-contact print on the basis of i-motif formation, the method comprising the steps of
providing to a surface of a substrate a solution with a bioconjugate according to claim 1, and
allowing the assembly of the bioconjugate in the i-motif or the i-motif related structure on the surface of the substrate to form the micro-contact print.

83. A liquid crystal device (LCD) comprising a bioconjugate according to claim 1.
